(19) United States
(12) Patent Application Publication
(10) Pub. No.: US 2018/0140302 A1
(43) Pub. Date: May 24, 2018

(54) Devices and Methods for Delivery of Materials into the Nose

(71) Applicant: CogENT Therapeutics, LLC, Hayward, CA (US)

(72) Inventors: Suresh Subraya Pai, Los Altos, CA (US); Celso J. Bagaoisan, Union City, CA (US); Scott Robert Sershen, Foster City, CA (US); Andrew Lawrence de Jong, College Station, TX (US)

(21) Appl. No.: 15/574,797

(22) PCT Filed: May 26, 2016
(86) PCT No.: PCT/US2016/034289

§ 371 Date: Nov. 16, 2017

Related U.S. Application Data
(60) Provisional application No. 62/218,955, filed on Sep. 15, 2015.
(60) Provisional application No. 62/167,214, filed on May 27, 2015.

(51) Int. Cl. A61B 17/12; A61F 13/26; A61F 13/12; A61B 17/24

(52) U.S. Cl. CPC A61B 17/12104; A61B 17/12159; A61B 17/1219; A61F 13/2005; A61F 13/126; A61B 17/24; A61F 13/26

(57) ABSTRACT

The various embodiments of the subject invention included herein provide devices and methods to deliver nasal packing material to a body lumen, cavity, or other anatomical structure along with methods of use of the same.

### CROSS REFERENCE TO RELATED APPLICATIONS

This application claims priority benefit to the filing date of U.S. Provisional Patent Application Ser. Nos. 62/167,214, filed on May 27, 2015, and 62/218,955, filed on Sep. 15, 2015, the disclosures of which applications are herein incorporated by reference in their entirety.

### BACKGROUND OF THE INVENTION

It is well established that there exist numerous clinical needs and applications that warrant and require the use of nasal packing materials. For example, in the context chronic pathologies such as rhinosinusitis or alternatively in the context of epistaxis.

Chronic rhinosinusitis (CRS) or inflammation of the mucosal lining of the nose and paranasal sinuses, is a condition that reportedly affects 39 million people each year accounting for greater than 22 million office visits and 250,000 emergency room visits per year in the United States. Inflammation of the mucosal lining of the paranasal ostia restricts the natural drainage of mucous from the sinus cavity through mucocilliary clearance resulting in chronic infections within the sinus cavity. Symptoms of chronic rhinosinusitis include extreme pain, pressure, congestion, and difficulty breathing. The first line of treatment for chronic rhinosinusitis is medical therapy including the administration of medications such as antihistamines, antibiotics and anti-inflammatory agents such as steroids. Patients that are unresponsive or refractory to this medical therapy typically are considered for surgical intervention to help relieve the symptoms of the condition. Functional endoscopic sinus surgery (FESS) is currently the most common type of surgery used to treat chronic sinusitis by remodeling the sinus anatomy via removal of mucosal tissue and bone. In a typical FESS procedure, an endoscope is inserted into the nose or nostril often along with a variety of other rigid, surgical instruments typically in a surgical operating room setting. These have traditionally included, but are not limited to the following tools: applicators, chisels, debriders, curettes, elevators, forceps, gouges, hooks, knives, saws, mallets, morselizers, needle holders, osteotomes, ostium seekers, probes, punches, backbiters, rasps, retractors, rongeurs, scissors, snares, specula, suction canulae and trocars. These instruments are then used to cut tissue and/or bone, cauterize, suction, debride etc. in order to remodel paranasal sinuses and adjacent anatomy sufficiently to restore outflow of mucus. FESS, which was developed as an alternative to open surgical incisions and procedures, encompasses the use of an endoscope along with the listed tools to minimize patient trauma. In spite of this, these surgical procedures produce mucosal surfaces that are raw and rough, and that have a propensity to continue bleeding and/or form post operative scars and adhesions. In this setting, nasal packing is employed post operatively today by ENT surgeons, Emergency room and Urgent Care physicians/staff, as well as primary care doctors to mitigate such issues. Said packing materials or nasal packs serve to mitigate local bleeding by absorbing local fluids (e.g. mucus, blood) and then stent tissue (i.e. physically separate tissue surfaces during the immediate post operative period when the mucosal surfaces are healing. The materials then biodegrade and run off via natural mucus outflow so as to limit patient discomfort at follow up related to painful removal of such materials.

Alternatively, epistaxis occurs as a result of known pathological conditions and often also via idiopathic pathways. Epistaxis (commonly known as nosebleed) is due to the rupture of blood vessels within the richly perfused, highly vascularized nasal mucosa. These ruptures may be spontaneous or sometimes initiated by trauma. Such nosebleeds impact a large portion of the human population (up to 60%). Hypertension, bleeding disorders and anticoagulant medication regimens add further complicating factors that tend to increase the duration of spontaneous epistaxis in many patients. Further, spontaneous epistaxis is very common in elderly people as their nasal mucosa (lining) is prone to becoming dry and thin and again their blood pressure tends to be higher. The combination of these factors makes the elderly subpopulation more prone to prolonged nosebleeds as their blood vessels are less able to constrict and control the bleeding.

The vast majority of nosebleeds occur in the anterior (or front) part of the nose or nasal compartment from the nasal septum. This area is richly endowed with blood vessels in the vicinity of the nasal anatomy known as Kiesselbach's plexus in a region also known as Little's area. Treatment of epistaxis spans a number of methods and devices that are directed to stopping blood flow to urge formation of clots. It is well understood that bleeding can be arrested via application of direct pressure or manual compression of the offending blood vessel with or without combination of said pressure with devices and/or medications and/or other procedures (e.g. mechanical and chemical cautery). For anterior epistaxis, application of digital compression (i.e. pinching of the soft fleshy part of the nose about the Little's area) provides an effective means to staunch bleeding and to promote blood clot formation. Further, local application of vasoconstrictive agents (e.g. epinephrine, oxymetazoline, phenylephrine etc.) have been shown to reduce the bleeding and bleeding time in benign cases of epistaxis. Ultimately, the aforementioned degradable (resorbable) nasal packing materials also provide another valuable treatment tool for epistaxis. Nasal packing is especially useful since the source of the bleeding (i.e. the exact location or nidus of the bleeding) is difficult to ascertain. Nasal packing acts as a space filler applying direct pressure or tamponade to the mucosa within the nasal compartment or cavity and reduces/eliminates the needs for exact specificity on the source of the bleeding.

While placement and utilization of nasal packing in the context of CRS and epistaxis provides benefits to patients and physicians, such materials (in general) are placed in a laborious manual sequence wherein the surgeon or other medical professional has to fold and/or grasp such materials with hand tools like bayonet forceps and then potentially use other instruments to manipulate and position the materials in desired anatomical positions. Subsequent to placement, said materials are then hydrated and/or often impregnated with medications using manual injections with syringes and needles and/or flushed with cannulas or other similar tools. The manual nature of implementing nasal packing in the clinical environment is inefficient and can be slow, counter to the need to treat bleeding rapidly. Another issue related to current nasal pack placement is that the target anatomy may be difficult to access using rigid, conventional instruments. For example, ENT surgeons that wish to place nasal packing in the frontal recess can have difficulties maneuvering these materials (some of which can be relatively bulky) in the tight, confined, and often angulated spaces where the packing is intended to be positioned. Further, when the materials are positioned correctly, they can be hard to hydrate or impregnate with desired amounts of water or medication since the leading edge of the material may be obscured from the line of sight and/or the reach of the source of the fluid or medication. Thus, there exists a need for improved delivery instruments or tools and methods to better navigate the nasal anatomy to facilitate rapid, efficient and accurate placement of nasal packing with or without subsequent hydration and/or impregnation with fluids such as water, saline or medication. Especially with epistaxis, the real time arterial bleeding and emergent nature of such bleeding provides further support for improved tools and methods to treat the condition.

### SUMMARY OF THE INVENTION

Described herein are devices and methods to deliver nasal packing material to a body lumen, cavity, or other anatomical structure along with methods of use of the same. The devices and methods may further be used to wet or hydrate the nasal packing material with a fluid before, during, and/or after placement of the nasal packing material in the desired anatomical location. The devices generally comprise an elongate member that holds or carries the material such as a tube or the like, a plunger or stabilization member to maintain the position of the nasal packing during delivery or placement, a handle, and/or a fluid infusion member optionally with one or more fluid delivery channels and/or lumens.

In one embodiment of the invention, the delivery device comprises an elongate member that has distal and proximal ends with at least one lumen extending through the length of the elongate member. The inner diameter of the elongate member may be sized to accommodate the nasal packing material of the invention and may be configured to have a constant inner lumen diameter or variable or tapered inner lumen diameter. The elongate member may be fabricated from metal and polymeric materials widely known in the art including, but not limited to, stainless steel, nickel, titanium, and alloys thereof, polyethylene, nylon, silicone, polyimide, acrylic, Pebax, polyurethane, PEEK, acetal, polycarbonate, polytetrafluoroethylene, combinations and copolymers thereof, composite materials and the like. The wall of the elongate member may have a constant or variable thickness, and may optionally comprise at least one lumen or channel. The elongate member may have a fixed or variable stiffness over the entire length or the portion of its length. The elongate member may also comprise articulating joints or segments (e.g. accordion, transverse cuts, hinges, etc.) that allow the shape (e.g. the angle or curvature) of the elongate member to be adjusted. The articulating joints or segments may be fabricated of materials that are different from the bulk of the elongate member, and may be located at a single or varied position along the length of the elongate member. For example, an articulating segment may allow the distal end of elongate member to bend from a relatively straight angle (i.e. generally 0°) to an angle of up to 120° from the longitudinal axis of the elongate member. The articulating segment may be located at a distance from the tip of the elongate member, preferably three (3) centimeters from the distal end of the elongate member. Alternatively, the elongate member may be fabricated with a specified angle incorporated into the elongate member at a specified distance from the distal end of the elongate member. For example, the distal tip may be angled at 0° to 20°, 20° to 40°, 40° to 60°, 60° to 80°, 80° to 100°, 100° to 120°, 120° to 140°, 140° to 160°, or 160° to 179° from the longitudinal axis of the main body of the elongate member. Preferable amounts of angulation are 0°, 70° and 120°, however other angles may be specified to suit the particular target application. The elongate member or segments thereof may be malleable such that the curvature, arc, angle, or shape of the elongate member can be set or adjusted to a desired configuration in an analog manner prior to use and then maintained in said configuration. Such a malleable, elongate member may comprise a single lumen or multiple lumens that can be plastically deformed to set the shape of the elongate member. Alternatively, the elongate member may comprise two or more lumens, one or more of which may contain a length of malleable rod or tube that can be plastically deformed to set the shape of the elongate member. The other lumen may remain open to accept the insertion of other tools or device components, provide a means for infusion of fluid (e.g. gas, liquid) or other materials through the elongate member, or the like. Alternatively, the lumen of a malleable tube can also provide the means for infusion of fluids or other materials.

The at least one lumen or channel may extend from the proximal end to the distal end of the elongate member, creating an end opening. Each individual lumen or channel may have a different shape in cross-section, including but not limited to elliptical (e.g. circular or oval), polygonal (e.g. convex, concave, regular, equilateral, equiangular, rectilinear, simple, combinations thereof, etc.), a combination of curved and straight segments, and the like. Alternatively, the at least one lumen or channel may terminate at a distance proximal to the distal end of the elongate member. In the case of an elongate member comprising multiple lumens or channels, each lumen may terminate at a uniform distance from the distal end of the elongate member or each lumen may terminate at a different distance from the distal end of the elongate member. Furthermore, the lumens or channels may be spaced radially about the elongate member in any configuration. For example, a circular elongate member with four lumens or channels may be designed such that the four lumens are located at approximately 0°, 90°, 180°, and 270° from the geometric center of the elongate member. In this example, the lumens or channels located at approximately 0° and 180° may terminate at the distal end of the elongate member while the lumens or channels located at 90° and 270° may terminate approximately one (1) centimeter proximal to the distal end of the elongate member. In the case of lumens residing within the elongate member, each of the at least one lumens may further comprise at least one hole or opening extending from the interior of the lumen to the interior and/or exterior wall(s) of the elongate member. These hole(s) may be spaced at any position or in any pattern along the length of the lumen. For example, a series of three holes may be located in four lumens of the prior example spaced equidistantly from each other and the distal end of the elongate member over a total length of approximately four (4) centimeters. It should be clear to one of skill in the art that the specific size, location, number, and pattern of holes in each individual lumen, and the number, size, locations, and cross-sectional shape of the lumen(s) within the elongate member may be chosen to suite the particular application of a given embodiment of the device of the invention. In yet another example, a device intended to deliver a large amount of fluid to wet or hydrate an approximately one (1) cm long nasal pack contained at the distal end of the elongate member may comprise eight (8) individual lumens arranged in an equiangular manner about the geometric center of the cross section of the elongate member. Each lumen may be of relatively large cross-sectional area with multiple, relatively large, hole(s) or opening(s) or communicating to the internal wall of the elongate member clustered in the distal, approximately one (1) centimeter of the elongate member. Alternatively, the use of channels that are open or in communication to the internal wall of the elongate member for a majority of the length of the elongate member may provide the same function (i.e. serves as fluid path). Permutations of the quantity and characteristics of the hole(s), lumen(s) and/or channel(s) present in the elongate member are simple extensions of the basic examples disclosed herein and are contemplated by this invention.

The elongate member may have a shape in cross section that is elliptical (e.g. circular or oval), polygonal (e.g. convex, concave, regular, equilateral, equiangular, rectilinear, simple, combinations thereof, etc.), a combination of curved and straight segments, and the like. The outer diameter and/or cross-sectional shape of the elongate member may be constant or variable over the length of the elongate member. For example, an elongate member of circular cross-section may have an outer diameter of six (6) millimeters over the majority of its length along with a conical taper over the distal two (2) centimeters to an outer diameter of three (3) millimeters at the distal tip of the elongate member. Alternatively, the distal tip of the elongate member may be configured to have a closed distal end, the wall of the closed distal end having cuts, serrations, scores and the like such that a second component that is advanced through the lumen of the elongate member can break open said closed, distal end of the elongate member via application of a set amount of force. For example, a nasal pack that is sensitive to fluids may be disposed within the elongate member's lumen or channel adjacent to the closed distal end. The closed distal end of the elongate member would protect the nasal pack from exposure to local fluids (i.e. blood, mucus or any fluids introduced to the field by the physician) upon insertion of the elongate member into the nasal cavity. When ready to place the nasal pack, the nasal pack is pushed with a sufficient amount of force such that the scorings, serrations or cuts on the distal end of the elongate member open and enable the pack to exit through the distal end of the elongate member. Furthermore, the distal end may have added features or may comprise different material(s) that makes the leading tip of the elongate member atraumatic. In another example, the distal end of the elongate member may be cut at an angle to direct the placement of a nasal pack or other material in a specified direction. Other permutations of the cross-sectional geometry of the elongate member over the length of the elongate member should be obvious to one of skill in the art and are contemplated by this invention.

The elongate member may further comprise a flange or other external features such as protrusions, bumps or the like that increase the external dimension of the elongate member over a length of the elongate member. This flange or other external feature may be an integrated part of the elongate member (e.g. formed through injection molding, machining, or another such technique known in the art) or, alternatively, an independent component that is temporarily or permanently joined or fixed to the elongate member using techniques known in the art including but not limited to bonding (e.g. adhesive bonding), welding, over-molding, threading/tapping, crimping, detents, combinations thereof, and the like. In the case of a flange or other external feature is an independent part, it may be fabricated from metals and polymer widely known in the art including, but not limited to stainless steel, nickel, titanium, and alloys thereof, polyethylene, nylon, silicone, polyimide, acrylic, Pebax, polyurethane, PEEK, acetal, polycarbonate, polytetrafluoroethylene, combinations and copolymers thereof, and the like. The flange or other external features may be located at any position along the length of the elongate member, but preferably at two (2) cm from the distal tip. For example, the flange or other external feature may be joined at the distal tip of the elongate member, zero (0) to one (1) centimeter proximal to the distal tip of the elongate member, one (1) to two (2) centimeters proximal to the distal tip of the elongate member, two (2) to three (3) centimeters proximal to the distal tip of the elongate member, and so on in half (0.5) centimeter or less increments over the length of the elongate member. Alternatively, the flange or other external feature may be adjustable such that its position within the elongate member can be changed depending on the specific application. The feature may have any shape and size that is conducive to the function of the delivery device of the invention. For example, a delivery device intended to stabilize the elongate member against the alar rim and/or columella of a patient's nose may have a feature that is semi-circular in shape and oriented perpendicular to the central axis of the elongate member, and sized to allow the feature to contact and interfere with the alar rim and/or columella when the elongate member is introduced into the nasal cavity of the patient. In this example the feature may be joined to the elongate member at a point proximal to the distal end of the elongate member to enable the distal end of the elongate member to advance a desired distance inside the nasal cavity before the feature contacts the alar rim and/or columella. Alternatively, the feature of this example may be comprised of two straight bars that extend away from the central axis of the elongate member in an orientation perpendicular to the longitudinal axis of the elongate member wherein the bars join the elongate member at opposite sides of the elongate member when viewed in cross-section. The bars may have any shape, thickness, length, radius, or complex curvature. Another embodiment of the feature may take the form of a lever that extends away from central axis of the elongate member and is located towards the proximal end of the elongate member. Such a lever can be envisioned as a means to allow a surgeon to advance, retract, or rotate the elongate member once it is inserted into the nasal cavity of the patient. While these examples described a few possible shapes and orientations of the feature, other shapes and or orientations are contemplated, including but not limited to elliptical disks, wedges, polygonal or other geometric shapes, arms or bars, spirals, combinations thereof, and the like. Furthermore, any of these feature shapes may comprise a constant or variable thickness and a linear, curved, or complex shape in transverse cross section. The use of multiple features as described herein located at different positions along the length of the elongate member is also contemplated.

The delivery device may additionally comprise at least one hollow or solid plunger that is moveably disposed within the lumen of the elongate member. The plunger may be fabricated from metals or polymers widely known in the art including, but not limited to, stainless steel, nickel, titanium, and alloys thereof, polyethylene, nylon, silicone, polyimide, acrylic, Pebax, polyurethane, PEEK, acetal, polycarbonate, polytetrafluoroethylene, combinations and copolymers thereof, and the like. The plunger may be free to move relative to a fixed elongate member, or alternatively, the elongate member may be free to move relative to a fixed plunger wherein said plunger acts as a stabilization member to maintain position of the nasal pack held within the lumen of the elongate member whilst the elongate member is retracted to expose the nasal pack. The plunger may further comprise additional extrusions and/or features that enable a surgeon to advance, retract, or rotate the plunger relative to the elongate member. In transverse cross section, the plunger may be rectangular, tapered, stepped, or a combination thereof, have a uniform or variable geometry along its length, and the like. In orthogonal cross section, the plunger may have a shape that is elliptical (e.g. circular or oval), polygonal (e.g. convex, concave, regular, equilateral, equiangular, rectilinear, simple, combinations thereof, etc.), a combination of curved and straight segments, and the like. The outer diameter and/or cross-sectional shape of the plunger may be constant or variable over the length of the plunger. For example, a cylindrical plunger moveably disposed within a cylindrical elongate member may further comprise a cylindrical tube or extrusion extending from distal face of the plunger that has a cross-sectional dimension that is less than that of the main body of the plunger. The distal end of the cylindrical tube may terminate proximal to, aligned with, or distal to the distal end of the elongate member. In this example, a nasal pack, gel, or other material may reside in the space between the outer diameter of the cylindrical tube and the inner diameter of the elongate member. Alternatively, the plunger may have a shape that matches or mates to the inner surface of the elongate member. For example, if the elongate member comprises a distal taper, the plunger may also comprise a distal taper appropriately dimensioned such that the distal end of the plunger is aligned with the distal end of the elongate member.

The plunger may further comprise at least one lumen or channel. Each individual lumen or channel may have a different shape in cross-section, including but not limited to elliptical (e.g. circular or oval), polygonal (e.g. convex, concave, regular, equilateral, equiangular, rectilinear, simple, combinations thereof, etc.), a combination of curved and straight segments, and the like. Alternatively, the at least one lumen or channel may terminate at distance proximal to the distal end of the plunger. In the case of a plunger comprising multiple lumens or channels, each lumen may terminate at a uniform distance from the distal end of the plunger or each lumen may terminate at a different distance from the distal end of the plunger. Furthermore, the lumens or channels may be spaced radially about the plunger in any configuration. For example, plunger with four lumens or channels may be designed such that the four lumens are located at 0°, 90°, 180°, and 270° from the geometric center of the plunger in cross section. In this example the lumens or channels located at 0° and 180° may terminate at the distal end of the plunger while the lumens or channels located at 90° and 270° may terminate one (1) centimeter proximal to the distal end of the plunger. In the case of lumens residing within the plunger, each of the at least one lumens may further comprise at least one hole or aperture extending from the interior of the lumen to the exterior wall of the plunger. These hole(s) may be spaced at any position or in any pattern along the length of the lumen. For example, a series of three holes may be located in four lumens of the prior example spaced equidistantly from each other and the distal end of the plunger over a total length of four (4) centimeters.

In yet another embodiment of the plunger of the delivery device, the plunger may comprise an extending member originating from the distal face of the plunger that has cross-sectional area that is less than the cross-sectional area of the main body of the plunger. The distal end of the extending member may terminate proximal to, in alignment with, or distal to the distal end of the elongate member. The extending member of the plunger may further comprise a lumen that is in communication with the plunger lumen and may extend to the distal end of the extending member. The wall defined by the inner surface and the outer surface of the extending member may also comprise one or more side holes or openings positioned along the length of the extending member, enabling fluid (e.g. gas, liquid) or other material to exit at the distal end opening and/or through the side hole(s) or opening(s). Alternatively, the distal end of the lumen of the extending member may be closed such that said fluid and/or other materials are delivered and exit the member exclusively via the said side hole(s) or opening(s). It should be clear to one of skill in the art that the specific size, location, number, and pattern of holes in each individual lumen, and the number, size, location, and cross-sectional shape of the lumen(s) within the plunger may be chosen to suit the particular application of a given embodiment of the device of the invention.

In yet another alternative embodiment of the invention, the at least one lumen used for fluid delivery of the extending member and/or plunger may comprise at least one valve disposed about the length of the lumen. This valve may be operative in an active or passive manner Such a valve would be particularly useful to prevent accidental or inadvertent fluid delivery or control of fluid delivery by the surgeon operator.

The delivery device may further comprise a handle fabricated from metal and polymeric materials widely known in the art including, but not limited to, stainless steel, nickel, titanium, and alloys thereof, polyethylene, nylon, silicone, polyimide, acrylic, Pebax, polyurethane, PEEK, acetal, polycarbonate, polytetrafluoroethylene, combinations and copolymers thereof, composite materials and the like. Differing embodiments of the handle may interact with the elongate member and/or plunger in a variety of ways. In one example, the handle may be sized and fabricated to comprise an actuator (e.g. a push button, lever, knob, gear, thumb slider switch or the like as known in the art) that is connected to the plunger and enables the surgeon to advance or as desired to retract the plunger within the elongate member. The proximal end of the elongate member may be joined to the handle. The plunger and/or elongate member may be sized such that when the plunger is fully retracted, a length of the lumen of the elongate member is open and can receive or be loaded with a portion of nasal packing or other material. Alternatively, the portion of nasal packing or other material may be preloaded into the delivery device, ready for delivery into a patient. Activating the actuator advances the plunger distally and drives the portion of nasal packing or other material out of the distal end of the elongate member. The handle may further comprise a stop or a detent or other feature that limits the distance the plunger can travel relative to the elongate member. In a second example, the handle may be sized and fabricated to comprise an actuator (e.g. a push button, level, knob, gear, thumb slider switch or the like as known in the art) that is connected to the elongate member and enables the surgeon to advance or retract the elongate member with respect to the plunger. The proximal end of the plunger in this example may be joined to the handle. The elongate member and/or plunger may be sized such that when the elongate member is fully extended, a length of the lumen of the elongate member is open and can receive at least a portion of nasal packing or other material. In some cases, the portion of nasal packing or other material may be preloaded into the delivery device, ready for delivery into a patient. In this example, activation of the actuator retracts the elongate member proximally and exposes the said portion of nasal packing or other material at the distal end of the delivery device. The handle may further comprise a stop or detent or other feature that limits the distance the elongate member can travel relative to the plunger.

A second embodiment of the invention comprises an elongate member and plunger as previously described, wherein the delivery device further comprises an movable, elongated solid rod or hollow support member extending distally from distal face of the plunger that has a cross sectional area that is less than the cross sectional area of the main body of the plunger. The elongated solid rod or hollow support member may be a separate part that has a distal end and proximal end, slidably coupled along the length of one of the plunger's lumens. The distal end of the elongated solid rod or hollow support member may terminate proximal to, in alignment with, or distal to the distal end of the elongate member. The proximal end of the elongated solid rod or hollow support member may terminate proximal of the proximal end of the plunger. In the case of a hollow support member, said member may have several holes or openings located between the distal end of its lumen and the distal end of the plunger positioned along the length of the hollow support member, enabling fluid (e.g. gas, liquid) or other material to exit at the distal end opening and/or through the side hole(s) or opening(s). Alternatively, the distal end of the lumen of the hollow support member may be closed such that said fluid and/or other materials are delivered and exit the member exclusively via the said side hole(s) or opening(s). An actuator located in the handle may be joined to the hollow support member to enable said member to advance or retract with respect to the plunger and the elongate member. In this embodiment, the proximal end of the hollow support member may comprise a port or similar means that facilitates attachment of a fluid source such that fluid can flow from the source, through the lumen of said member, and exit via the end hole lumen and/or the side holes or openings. Further, said hollow support member may be retracted proximally independent of the plunger and/or elongate member after the portion of nasal packing or other materials has been wetted or hydrated.

In yet another alternative embodiment of the delivery device of the invention, the elongate member may be configured to include a distal portion that comprises a larger luminal space that receives a portion of nasal packing or other material. The portion of the elongate member having a smaller luminal space may be configured to coaxially receive a plunger. The said plunger further comprises a flange at or along its distal segment sized to slidably fit within the larger, distal lumen portion of the elongate member and to interfere with the luminal transition of the smaller proximal portion to the larger, distal portion of the elongate member. The flange of the plunger would provide a stop to maintain the position of the portion of nasal packing or other material (i.e. reduce or eliminate proximal pullback of the nasal packing or other material) as the elongate member is retracted proximally during delivery of said packing or other material.

The proximal end of the plunger may be reversibly or irreversibly joined to the fluid reservoir. A reversible joint may be achieved using techniques known in the art, including but not limited to friction fittings (e.g. an elastic tube or sheath fitting over a tapered port on the reservoir), barbed tube fittings, compression tube fittings, slip luer fittings, luer lock fittings, quick-disconnect fittings, valved quick-disconnect fittings, threaded/tapped fittings, push-to-connect fittings, and the like. A reversible joint can be used when an external reservoir is the source of the fluid. Alternatively, a permanent or irreversible joint can be used when an internal reservoir is the source of the fluid. Any such joint between a reservoir and the proximal end of the plunger may further comprise o-rings, seals, gaskets, and the like fabricated from materials known in the art such as silicone rubber, natural rubber, nitrile butadiene rubber, polyurethane, neoprene, fluoroelastomers, and the like to contain the fluid in the desired flow path. In one example of this embodiment of the invention, the proximal end of the plunger comprises an elastic connector (e.g. a silicone rubber connector) concentrically aligned with and joined to the outer diameter of the plunger such that the proximal end of the plunger partially resides within the distal portion of the lumen of the elastic connector. The elastic connector is of a length that is sufficient to receive and secure the tapered portion (e.g. a nozzle tip component or section) of a fluid reservoir (e.g. a squeeze bottle containing a saline solution or medication). The elastic connector may be exhibit properties that it allow it to comply and shape itself about the nozzle tip of the reservoir. For example, the elastic properties (i.e. durometer) and the luminal dimensions (i.e. the inner diameter) of the elastic connector could be optimized to effectively seal and/or be secured around the tapered portion of said reservoir during use. Alternatively, if the tapered portion or nozzle of said reservoir includes one or more longitudinal channels that are occasionally included on commonly used medication squeeze bottles, then the elastic connector may further comprise an integrated sealing means at the base of the connector such as o-ring or the like. In this embodiment, the sealing means or o-ring could be sized such that it can contact and form a seal about the distal tip of the reservoir nozzle when it is fully inserted into the elastic connector. Compressing or squeezing the flexible bottle forces the saline or medication through the lumen of the plunger and out of the holes and/or apertures of the plunger extrusion.

One method of use for this embodiment of the invention is to load a nasal packing material or stent (or other material to be delivered into the nasal cavity) into the distal end of the elongate member, and attach a squeezable bottle to the proximal end of the plunger. The distal portion of the device is inserted within the nostril or anterior nasal compartment of a patient until the flange located on the distal portion of the elongate member physically contacts the alar rim or columella of the nose. In this method, the plunger is then continued to be advanced distally by applying force to the proximal portion of the device of the invention (e.g. at the reservoir), until the packing material or stent or other material is substantially exposed. At this point a significant portion of the nasal packing or stent or other material has been pushed past the distal end of the elongate member and is resident inside the nose and/or nasal cavity of the patient. The reservoir or squeezable bottle is then compressed or squeezed to drive or deliver fluid (saline or medication) from said reservoir into the nasal packing or other material and within the nose or nasal cavity.

In a third embodiment of the invention, the delivery device comprises an elongate member, plunger, and handle as previously described wherein the plunger further comprises a lumen extending from its proximal to distal end. Further, the delivery device comprises a fluid delivery member comprising an elongate tube coaxially fabricated from materials known in the art including, but not limited to stainless steel, nickel, titanium, and alloys thereof, polyethylene, nylon, silicone, polyimide, acrylic, Pebax, polyurethane, PEEK, Delrin, acetal polymers, polycarbonate, polytetrafluoroethylene, combinations and copolymers thereof, and the like. The distal end of said fluid delivery member may further comprise a support member to provide stiffness to the distal segment of the fluid delivery member. The support member may be pre-shaped or alternatively it may possess malleable properties that allow it and the overall fluid delivery member distal segment to be shaped as required. Said support member may be fabricated from materials known to the art including, but not limited to stainless steel, nickel, nickel titanium, titanium, and alloys thereof, and the like. The support member may take a form commonly known in the art, including but not limited to a sinus seeker, a guide wire as commonly used in cardiovascular and sinus surgeries, and the like. The support member may be sized such that fluid may be injected around the support member when the support member is disposed within the lumen used for fluid delivery. The support member may be fixed in translation and/or rotation with respect to any or all of the other components of the delivery device, or the support member may be free to translate or rotate with respect to any of all of the other components of the delivery device. For example, the support member may extend a distance beyond the distal end of the fluid delivery member, elongate member, and the plunger, comprise an atraumatic tip (e.g. rounded, ball, beveled, coiled, spring, combinations thereof, and the like), and be fixed in translation and rotation with respect to the other components of the device. With this, it is obvious that the elongate member that is coaxial to the fluid delivery member may also possess similar malleable properties or capability and could be shaped along with the under portions of the delivery device and its payload (i.e. nasal packing material or stent or other material to be delivered). The fluid delivery member may be substantially coaxially aligned with the elongate member and plunger, and sized such that there is a clearance fit between the inner wall of the plunger lumen and the outer wall of the fluid delivery member. The fluid delivery member may be fixed or free to move with respect to translational movement relative to the elongate member. In one preferable embodiment, the fluid delivery member and elongate member are fixed relative to each other and the plunger can translate in the proximal and/or distal directions with respect to the fluid delivery member and the elongate member. In another preferable embodiment, the fluid delivery member and plunger are fixed relative to each other and the elongate member can translate in the proximal and/or distal directions with respect to the plunger and fluid delivery member. The fluid delivery member may further comprise at least one lumen extending from the proximal to distal ends of the fluid delivery member. The lumen may further extend through the distal end of the fluid delivery member terminating at its distal end with an end opening, or alternatively, the lumen may be closed at its distal end. The fluid delivery member may further comprise at least one hole or other aperture extending from its inner wall to its exterior wall. These hole(s) may be spaced at any position or in any pattern along any portion or segment of the length of the fluid delivery member. The proximal end of the fluid delivery lumen may be reversibly or irreversibly joined to a fluid reservoir as previously described.

As mentioned previously in this specification, a preferable embodiment of the invention comprises an elongate member, a plunger, a fluid delivery member, a handle, and a fluid reservoir, wherein the fluid delivery member and plunger are fixed relative to each other and the elongate member can translate in the proximal and/or distal directions with respect to the plunger and fluid delivery member. The elongate member further comprises a feature to enable the user to advance or retract the elongate member with respect to the plunger and fluid delivery member, such as an extrusion that extends from the outer surface of the proximal portion of the elongate member and through a slot in the handle. Other means of advancing or retracting the elongate member (e.g. gears, ratchets, ball and spring mechanisms, threads/taps, and the like) should be obvious to one of skill in the art and are contemplated herein. The plunger is sized such that the distal end of the plunger is recessed a desired distance from the distal end of the elongate member when the elongate member is fully advanced in the distal direction. The fluid delivery member is sized such that the distal end of the fluid delivery member is substantially aligned with the distal end or tip of the elongate member when the elongate member is fully advanced in the distal direction. The resulting space, defined by the distance between the outer surface of the fluid delivery member lumen and the inner surface of the elongate member and the distance extending between the distal end of the elongate member and the distal end of the plunger, may receive a nasal packing material or stent (or other material to be delivered into the nasal cavity and/or paranasal sinuses). It is contemplated that a nasal packing or stent or other material to be delivered may be loaded by a user into this space at the time of use or alternatively could be loaded into the space during manufacture/fabrication of the delivery device of the invention. The outer dimension of the elongate member may be chosen to suit the target anatomy. For example, a delivery device intended to position a nasal packing material or stent or other material in the ethmoid sinus(es) may be larger than a delivery device intended to position a nasal other material into the frontal sinus ostium. Likewise, the length of the space defined by the distance between the distal end of the elongate member (in the fully distally advanced position) and the distal end of the plunger may be lengthened or shortened based on the target anatomy. The elongate member, and the means of actuation of the elongate member, may be chosen such that the distal tip of the elongate member is positioned substantially even with or slightly proximal to the distal tip of the plunger when the elongate member is fully retracted in the proximal direction. The proximal end of the fluid delivery member may be reversibly or irreversibly joined to the fluid reservoir as previously described, and the distal portion of the fluid delivery member comprises holes or apertures between the fluid delivery member lumen and the other surface of the fluid delivery member.

One method of use for this preferred embodiment of the invention is to insert the distal portion of the device into the nostril of a patient until the distal portion of the elongate member reaches and/or cannulates the desired target anatomy (e.g. the frontal sinus recess or sinus ostium, the maxillary sinus or sinus ostium, an ethmoid sinus or sinus ostium, the sphenoid sinus or sinus ostium, or the like). The elongate member is retracted proximally to expose the nasal packing or stent or other material to or within the desired target anatomy, after which a user selected fluid may be delivered as desired to the interior of the material or stent and the surrounding anatomy via the lumen of the fluid delivery member.

The delivery devices of the invention may further comprise markers to enable the surgeon to determine the position of the device, or a component of the device, with respect to the anatomy of the patient. These markers may include visual indicators such as colored bands, radiologic indicators such as radio-opaque metals, alloys, and other materials, emitters or receivers for use in optical or electromagnetic image guidance systems (e.g. the Fusion ENT Navigation System from Medtronic Xomed, the InstaTrak System from General Electric, etc.), light emitting components for transdermal illumination, and the like.

The delivery device of the invention may deliver a variety of payloads with differing physical structures to the target anatomy, including but not limited to stents (self expanding and non-self expanding), nasal packing, gels, sponges, gauze, foams, hydrogels, xerogels, aerogels, particles, microparticles, nanoparticles, slurries or collections of particles, liquids of varying viscosity, solids, combinations thereof, and the like. The payloads may be biodegradable or non-biodegradable in nature. The payloads may be fabricated from materials known to the art, including but not limited to poly(ethylene glycol), poly(ethylene oxide), poly(vinyl alcohol), capralactone urethanes, poly(urethanes) and poly(urethane) derivatives, poly(allyl alcohol), poly(vinylpyrrolidone), poly(alkylene oxides), poly(oxyethylated polyols), poly(ethyleneimine), poly(allylamine), poly(vinyl amine), poly(aminoacids), poly(ethyloxazoline), poly(ethylene oxide)-co-poly(propylene oxide) block copolymers, poly(glycolic acid), poly(lactic acid), copolymers of poly(lactic acid) and poly(glycolic acid), poly(orthoesters), polysaccharides (e.g. chitin, chitosan, hyaluronic acid, the family of chondroitin sulfates, heparin, keratan sulfate, glycogen, glucose, amylase, amylopectin and derivatives thereof), carbohydrates, oligopeptides, polypeptides, caprolactones, carboxymethylcellulose, stainless steel, nickel, titanium, biologically derived polymers such as collagen, elastin, and the like, combinations, copolymers, and alloys thereof, and the like. The payload may be a material formulated as disclosed in co-pending U.S. patent application 61/259,564, the disclosure of which is incorporated herein by reference in its entirety. The payload may be active, in that one or more characteristics of the payload are altered after it is positioned at the target anatomy, or passive, in that the characteristics of the payload do not undergo significant change after it is positioned at the target anatomy. Examples of active payloads include, but are not limited to those that change shape via self-expansion after delivery and those that change shape due to absorption or adsorption of fluid after delivery, payloads that release drugs or therapeutic agents after delivery, payloads that change shape or form due to induction of electric current, pH or temperature responsive materials, combinations thereof, and the like.

The delivery device of the invention may be used to deliver fluid to the payload or surrounding anatomy (e.g. in the nasal cavity or within the sinuses or the like) before, during, or after delivery of the payload itself to the target anatomy. Fluids of interest include, but are not limited to water, saline, solutions of drugs or other therapeutic agents, probiotics, prebiotics, combinations thereof, and the like. Therapeutic solutions may comprise at least one therapeutic agent and an appropriate buffer solution. The therapeutic agents may include, but are not limited to anti-inflammatory agents, anti-allergens, anti-cholinergic agents, antihistamines, anti-infectives, anti-platelet agents, anti-coagulants, anti-thrombic agents, anti-scarring agents, anti-proliferative agents, chemotherapeutic agents, anti-neoplastic agents, decongestants, healing promoting agents and vitamins (for example, retinoic acid, vitamin A, depaxapanthenol, vitamin B and their derivatives), hyperosmolar agents, immunomodulators, immunosuppressive agents, and combinations and mixtures thereof as disclosed in U.S. Pat. No. 8,585,730, incorporated herein by reference in its entirety. Of particular interest are steroidal anti-inflammatory compounds such as budesonide, cloprednol, cortisone, fluticasone propionate, methylprednisolone, mometasone furoate, prednisolone, triamcinolone, triamcinolone acetonide, triamcinolone benetonide, triamcinolone hexacetonide or any triamcinolone derivatives, and the like, as well as decongestants such as epinephrine, pseudoephedrine, oxymetazoline, phenylephrine, tetrahydrozolidine, xylometazoline, and the like.

One preferable method of use of the invention herein is to shape the distal portion of the delivery device as desired depending upon the targeted anatomy, to attach a fluid reservoir to the proximal portion of the delivery device, to insert said device via the nose into the nasal cavity and to position near or within the paranasal sinuses, to substantially expose the payload of the device, to deliver user selected fluids to the payload and/or surrounding anatomy via the fluid delivery member and/or the lumen of the plunger, and then to remove the delivery device from the patient. In this method, the user delivered fluid could be delivered in such volume that it is available for absorption into the nasal mucosa and/or the delivered payload after the delivery device is removed thereby allowing the fluid an opportunity to dwell for a more extended period of time. In this embodiment of the method of use of the delivery device of the invention, the user selected fluid being delivered may further comprise an initial injection of water or saline followed by an injection of a medication (e.g. a steroid or other medication in suspension as previously described). It should be obvious that this sequence could also be performed in reverse wherein the medication is delivered first followed by saline or water and furthermore the fluids may be delivered via the same or different lumens. It should also be obvious that the sequence of fluid delivery could include more than two fluids as described herein.

Alternatively, another preferable method of use of the invention herein is to shape the distal portion of the delivery device as desired depending upon the targeted anatomy, to attach a fluid reservoir to the proximal portion of the delivery device, to insert said device via the nose into the nasal cavity and to position near or within the paranasal sinuses, to deliver user selected fluids to the payload and/or surrounding anatomy, to substantially expose the payload of the device, to deliver more user selected fluids if desired to the payload and/or surrounding anatomy, and then to remove the delivery device from the patient. The sequence of fluid injection and the specific type of fluid selected by the user for injection may be performed as previously described.

In this method, the user delivered fluid could be delivered in such volume that it is available for absorption into the nasal mucosa and/or the delivered payload after the delivery device is removed thereby allowing the fluid an opportunity to dwell for a more extended period of time. Moreover, the payload (e.g. nasal packing material) may substantially plug or occlude the opening of the nasal cavity or air cell trapping any user delivered fluids posterior or distal to it and in doing so providing a fluid source to the payload. The payload would then act as a wick for the fluid and facilitate delivery of said fluid to a more substantive surface area of nasal mucosa.

Another embodiment of the invention comprises a footplate, a handle, and at least one tine or prong. In a first example, the embodiment comprises two tines. The tines may be fabricated from materials known to the art including, but not limited to, aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like. The tines may be elongate members wherein the largest cross sectional measure is less than the length of the tines, and may be arranged generally parallel to each other such that a packing material may be placed and held in the space between the tines. The distal ends of the tines may be formed into an atraumatic shape. The tines may be pre-formed such that each tine applies an inward force to the packing material to aid in fixing the position of the packing material relative to that of the tines. The packing material will be assumed to have a generally rectangular geometry, with a length that is greater than its height, and a height that is greater than its depth. It should be clear to one of skill in the art that the packing material may have any number of geometries and the number and orientation of the tines may be adapted to securely hold the packing material relative to the tines. The tines may be independent of each other or may be joined at some point along their lengths to a common elongate member (e.g. as in a tuning fork, forceps, and the like). The tines may also have a fixed or variable cross-sectional shape over the length of each tine. Furthermore, the tines may be identical in shape, symmetric, or asymmetric to each other. The distal portions of the tines are open to accept a packing material and the proximal end of the tines are joined to a link member. The link member is slidably disposed within a handle. The handle may be fabricated from materials known to the art, including but not limited to, aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like. The handle may be fabricated using means known to the art including but not limited to machining, stamping, injection molding, combinations thereof, and the like. The handle may be ergonomically shaped, contoured, etched, and the like to provide a comfortable, secure grip for the user. The handle may further comprise a slot that is generally aligned with the longitudinal axis of the handle and at least generally parallel to the tines. A connecting element may extend from the common elongate member, through the slot in the in the handle, and terminate in an actuator such as a button, slider, knob, or the like that enables a user to slide the tines proximally or distally with respect to the handle. The actuator may be ergonomically shaped, contoured, etched, and the like to provide a comfortable, secure grip for the user. The slot may further comprise features that aid in holding the tines in any one of a number of positions along the length of the slot. For example, a set of living hinges may preferentially hold the tines in a fully-distal position until a threshold force is applied to the actuator in the proximal direction. Likewise, another set of living hinges may perform the same function when the tines are in the fully proximal position. The features may be reversible, such that the tines may be cyclically advanced and retracted, or one or more of the features may be irreversible. In an example of the latter, a feature may be incorporated into the slot and/or handle that prevents the tines from moving distally once a threshold amount of retraction (i.e. proximal translation) from an initial distal position has occurred. The footplate of the embodiment may be fabricated from materials known in the art, including but not limited to aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like. The footplate may be positioned towards the distal end of the device and located between the generally parallel tines. Alternatively, the footplate may be sized such that the cross section of the footplate is larger than the tines, and further comprise through holes or cut-outs that enable the tines to pass through the footplate. The footplate is preferentially similar in area to the cross section of the packing material, but other sizes and shapes are contemplated. The proximal end of the footplate is connected to an additional elongate member using methods known to the art, including but limited to adhesive bonding, welding, ultrasonic welding, overmolding, threading and tapping, mechanical fixation, friction or interference fits, combinations thereof, and the like. The additional elongate member may be fabricated from materials known to the art, including but not limited to aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like. The proximal end of additional elongate member may be connected to the handle using methods known to the art, including but not limited to adhesive bonding, welding, ultrasonic welding, overmolding, threading and tapping, mechanical fixation, friction or interference fits, combinations thereof, and the like. Alternatively, the footplate and additional elongate member may be a single contiguous structure fabricated using methods known to the art including, but not limited to, injection molding, stamping, machining, combinations thereof, and the like. The additional elongate member may be sized such that it does not interfere with the translation of the tines with respect to the handle. The combined length of the footplate and additional elongate member is such that the distal end of the tines extends past the distal face of the footplate. While the tines may extend any distance beyond the distal face of the footplate, they preferentially extend beyond the distal face of the footplate a length that is equal to or less than that of the packing material. For example, the tines may extend beyond the distal face of the footplate a distance equal to 90%-100% of the length of the packing material, a distance 80%-90% of the length of the packing material, a distance 70%-80% of the length of the packing material, a distance 60%-70% of the length of the packing material, a distance 50%-60% of the length of the packing material, a distance 40%-50% of the length of the packing material, a distance 30%-40% of the length of the packing material, a distance 20%-30% of the length of the packing material, a distance 10%-20% of the length of the packing material, or a distance 1%-10% of the length of the packing material. Furthermore, the tines and additional elongate member may be fabricated from a malleable material to enable the angle and orientation of the packing material held within the tines to be adjusted as desired by the user. While the preceding description uses an embodiment comprising two tines for ease of explanation, however, it should be clear to one of skill in the art that any number of tines or prongs may be incorporated within the scope of the invention. All of the stated considerations may be applied to the exemplary embodiments described in FIGS. 9 through 15.

Alternatively, the tines and the footplate/additional elongate member may be enclosed within a shell, case, tubing, or the like. The shell, case, or tubing may be rigid or malleable, and may have any number of geometries, including but not limited to straight (or linear), curved, combinations thereof, and the like. For example, the shell, case, or tubing may comprise an at least two substantially straight lengths joined by a chicane or S-shaped segment to form a shape substantially similar to a bayonet forceps. In the instances where the shell, case, or tubing is not linear, at least one portion of the link member may be sufficiently flexible such that it can translate within the shell, case, or tubing without binding. For example, the link member may be fabricated in part from materials known to the art including, stainless steel, nickel-titanium alloy, nylon or polyamide, PEEK, polydioxanone, catgut, polylactic acid, polyglycolic acid, PLGA, silk, polypropylene, polyester, combinations or alloys thereof, and the like. In one example, the tines and the footplate/additional elongate member are enclosed within a length of stainless steel tubing that is bonded to the handle using means known to the art, including but not limited to adhesive bonding, welding, ultrasonic welding, overmolding, threading and tapping, mechanical fixation, friction or interference fits, combinations thereof, and the like. The length of the tubing is such that the distal face of the footplate is aligned with or distal to the distal end of the tubing. In another example, the footplate/additional elongate member is omitted from the device of the invention and is replaced by an elongate member further comprising lumens that are numbered, oriented, and sized to accept the tines. In this case the distal end of the elongate member serves the same function as the footplate. In yet another example, the body of the handle may extend in the distal direction to encompass at least a portion of the tines and footplate/additional elongate member. All of the stated considerations may be applied to the exemplary embodiments described in FIGS. 9 through 15.

This embodiment of the invention may be used to deliver a packing material into an anatomical structure such as the middle meatus. In such a method of use, the actuator is adjusted to the distal-most position within the slot on the handle and a packing material is inserted between the tines. The device of the invention is inserted into the nasal cavity of a patient via the nostril and the distal end of the device containing the packing materials is advanced into the middle meatus. The actuator is then retracted proximally, translating the tines in the proximal direction. The tines slide over the outer surfaces of the packing material and release the packing material into the middle meatus once the distal end of the tines passes the proximal end of the packing material. In cases where friction or another binding force exists between the tines and the packing material and drags the packing material in the proximal direction as the tines are retracted, the proximal face of the packing material contacts the distal face of the footplate as the tines are retracted and is prevented from translating further in the proximal direction.

Another embodiment of the invention further comprises an automated retraction feature such that when a button or other actuator is depressed, the tines retract to a fully proximal position. In some cases the automated retraction feature may augment or replace the manual retraction feature previously described. In one example, the feature comprises a spring disposed within the handle and located distal to the common elongate member, a disposed within the handle and located proximal to the common elongate member, and a means of moving the pin. In the locked position, the common elongate member is in a fully distal position within the handle (compressing the spring) and the pin is extended proximal to the common elongate member, holding the common elongate member in place. Moving the pin out of the path of the common elongate member allows the spring to expand, push the common elongate member proximally within the handle, and retract the tines proximally to release the packing material. The speed and force of the retraction may be modified by using springs of different material and/or physical characteristics (e.g. unloaded length, spring constant, wire thickness, wire shape, etc.). Alternatively, more complex systems such as the use of one or more constant force springs to may be employed to further modify speed and force of the retraction. While this example describes an exemplary spring-loaded retraction mechanism, other means of enacting an automated retraction mechanism will be clear to one of skill in the art. All of the stated considerations may be applied to the exemplary embodiments described in FIGS. 9 through 15.

Another embodiment of the invention comprises two tines joined at their distal ends to a link member, a handle, an actuator, a length of tubing, and a flexible linkage. The longitudinal axis of the tines is offset from the longitudinal axis of the handle (e.g. as seen in bayonet forceps). The length of tubing may be malleable or rigid. For example, a malleable tubing may comprise multiple lumens, one of which further comprises a deformable wire that is used to set the shape of the tubing. A rigid tubing may be generally fixed in shape and unable to substantially change shape when subjected to force. The tubing may be comprised of materials known to the art including, but not limited to, aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like. In this embodiment, the distal end of the tubing further comprises a footplate of a size and shape that will interfere with and prevent the proximal movement of a packing material held within the tines. The footplate further comprises thru-holes that are sized, shaped, and located to accept the tines and allow free movement of the tines in the proximal and distal direction. A segment of the tubing located between the distal and proximal ends is curved and/or shaped such that the distal end of the tubing is generally aligned with the longitudinal axis of the tines and the proximal end of the tubing is generally aligned with the longitudinal axis of the handle. An example of such a shape is a chicane or a short, shallow, S-shaped turn. The proximal end of the tubing is joined to the handle using means known to the art including, but not limited to, adhesive bonding, welding, ultrasonic welding, overmolding, threading and tapping, mechanical fixation, friction or interference fits, combinations thereof, and the like. The lumen of the tubing is in communication with the interior of the handle. The tines are slidably disposed within the distal portion of the tubing such that the distal ends of the tines exit the thru-holes of the footplate and extend distally beyond the footplate. The tines are of a size that permits the distal portions of the tines that extend beyond the tubing to adequately grasp a packing material. The proximal ends of the tines are joined to a link member at a point within the tubing distal to the curved and/or shaped segment. The tines and the link member may be shaped from a contiguous material (e.g. stamped out of stainless steel), or alternatively, the tines and the link member may be individual components that are joined together using means known to the art including, but not limited to, adhesive bonding, welding, ultrasonic welding, overmolding, threading and tapping, mechanical fixation, friction or interference fits, combinations thereof, and the like. The distal end of the flexible linkage is joined to the proximal end of the link member using means known to the art including, but not limited to, adhesive bonding, welding, ultrasonic welding, overmolding, threading and tapping, mechanical fixation, friction or interference fits, combinations thereof, and the like. The flexible linkage extends proximally within the tubing and is sized such that the flexible linkage can freely move within the tubing. The proximal end of the flexible linkage is joined to an actuator located within the interior of the handle. In one example, the actuator is a post that is connected to an external slider located on the exterior of the handle via a slot cut into the surface of the handle. Moving the slider in the proximal direction pulls the flexible linkage proximally, which in turn retracts the tines in the proximal direction. In another example, the actuator is a rotating spool connected to a knob on the exterior of the handle via a hole cut into the surface of the handle. Turning the knob winds the flexible linkage around the spool, which in turn retracts the tines in the proximal direction. While these two actuators have provided exemplary embodiments, it should be clear to one of skill in the art that other means of actuating the tines are contemplated. The handle may further comprise features that aid in maintaining the actuator in a given position or positions. For example, ratcheting mechanisms may be employed to allow only clockwise turning of a knob and spool style actuator. In another example, detents and living hinges may be employed to prevent premature proximal motion of a slider style actuator in the proximal direction. Furthermore, the previously described means of automating the action of retracting the tines are applicable to this embodiment of the invention. All of the stated considerations may be applied to the exemplary embodiments described in FIGS. 9 through 15.

Another embodiment of the invention comprises an elongate member, an extension line, an expandable member, and a connection port. The elongate member may be fabricated of materials known to the art, including but not limited to aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like. The elongate member has distal and proximal ends and may be hollowed out such that the distal end is closed and the proximal end is open. The distal end of the elongate member may further be formed into an atraumatic shape including but not limited to tapered, curved, rounded, hemispherical, and the like. The elongate member may further comprise at least one inflation port allowing communication between the interior of the elongate member and the external environment. The at least one inflation port may be located at any position (radially or longitudinally) along the elongate member. It is preferable for the elongate member to have at least two inflation ports located equidistantly from each other (radially) and in the distal portion of the elongate member. For example, an elongate member with four inflation ports would preferentially have the ports located at 0°, 90°, 180° and 270° about the perimeter of the elongate member. Alternatively the inflation ports may be staggered or spaced longitudinally and/or radially. The elongate member may further comprise a flange or other external features such as protrusions, bumps or the like that increase the external dimension of the elongate member over a segment of the elongate member. This flange or other external feature may be an integrated part of the elongate member (e.g. formed through injection molding, machining, or another such technique known in the art) or, alternatively, an independent component that is temporarily or permanently joined or fixed to the elongate member using techniques known in the art including but not limited to bonding (e.g. adhesive bonding), welding, over-molding, threading/tapping, crimping, detents, combinations thereof, and the like. In the case of a flange or other external feature is an independent part, it may be fabricated from metals and polymer widely known in the art including, but not limited to stainless steel, nickel, titanium, and alloys thereof, polyethylene, nylon, silicone, polyimide, acrylic, Pebax, polyurethane, PEEK, acetal, polycarbonate, polytetrafluoroethylene, combinations and copolymers thereof, and the like. The flange may be preferentially located towards the proximal portion of the elongate member.

The extension line is a flexible elongate member with distal and proximal ends that further comprises at least one lumen extending the length of the extension line. The extension line may be fabricated from materials known in the art, including but not limited to, aliphatic polyamides, fluorinated ethylene propylene, nylon, perfluoroalkoxy (e.g., Teflon®), polyether block amide (Pebax®), polyetheretherketone (PEEK), polyethylene, polytetrafluoroethylene (PTFE), polypropylene, polyurethane, polyvinylchloride, natural rubber, nitrile rubber, silicone rubber, combinations and copolymers thereof, and the like. The distal end of the extension line is joined to the proximal end of the elongate member using means known to the art including, but not limited to, adhesive bonding, welding, ultrasonic welding, overmolding, threading and tapping, mechanical fixation, friction or interference fits, combinations thereof, and the like. The proximal end of the extension line is joined to a connection port using means known to the art including, but not limited to, adhesive bonding, welding, ultrasonic welding, overmolding, threading and tapping, mechanical fixation, friction or interference fits, combinations thereof, and the like. The joints between the elongate member, extension line, and connection port are such that the parts are in fluid communication with each other. Fluid injected into the connection port will flow down the extension line, into the elongate member, and out of the inflation ports. The connection port may be comprise standard medical couplings including, but not limited to, male or female luer-locks (fixed or rotating), male or female luer-slips, male or female luer-activated valves, quick-disconnect fittings, hose barbs, internally threaded fittings, externally threaded fittings, flexible tubing, and the like. The connection port is preferably a female luer-activated valve. The fittings may be fabricated from materials known to the art including, but not limited to, polycarbonate, polyethylene, polyolefin, polypropylene, polytetrafluoroethylene, polysulfone, polyvinylchloride, polyoxymethylene (Delrin®)), brass, stainless steel, nylon, perfluoroalkoxy (e.g. Teflon®), natural rubber, nitrile rubber, silicone rubber, combinations thereof, and the like.

The expandable member may be fabricated from materials known to the art including, but not limited to, aliphatic polyamides, fluorinated ethylene propylene, nylon, perfluoroalkoxy (e.g., Teflon®), polyether block amide (Pebax®), polyetheretherketone (PEEK), polyethylene, polytetrafluoroethylene (PTFE), polypropylene, polyurethane, polyvinylchloride, natural rubber, nitrile rubber, silicone rubber, combinations and copolymers thereof, and the like. In one embodiment, the expandable member is preferably fabricated from an elastomeric material. The expandable member may be formed into an elongated tubular shape with a distal closed end and an open proximal end. The distal closed end may further be formed into an atraumatic shape including but not limited to tapered, curved, rounded, hemispherical, and the like. The elongate member is disposed within the expandable member such that the distal end of the elongate member abuts the distal internal surface of the expandable member. The proximal end of the expandable member is preferably located proximal to the proximal-most injection port of the elongate member. The proximal end of expandable member is joined to the elongate member using means known to the art including, but not limited to, adhesive bonding, welding, ultrasonic welding, overmolding, threading and tapping, mechanical fixation, friction or interference fits, combinations thereof, and the like such that the expandable member is sealed to the elongate member. The distal end of the expandable member may be sealed to the distal end of the elongate member, or it may be free floating (i.e. not joined to the distal end of the elongate member). A packing material may be disposed about the expandable member. The packing material may be shaped into a hollow cylinder by forming a tube from a flat sheet of packing material by rolling the sheet around a mandrel and joining the edges and/or portions of the outer surfaces of the sheet to each other using means known to the art including but not limited to adhesive bonding, welding, fusing, ultrasonic welding, suturing or tying, mechanical interlocks (e.g. dovetail joints, tongue and groove joints, box joints, etc.), combinations thereof, and the like. Alternatively, the packing material may be fabricated in a tubular form (e.g. cast and cured in a tubular mold). It should be clear to one of skill in the art that multiple means of fabricating a packing material that can wrap around the expandable member. In some cases, the packing material may optionally be secured to the expandable member using means known to the art, including but not limited to, adhesive bonding, welding, ultrasonic welding, friction or interference fits, combinations thereof, and the like. Preferably, the packing material is secured about the expandable member via an interference or friction fit between the inner surface of the packing material and the outer surface of the expandable member. Furthermore, the flange of the elongate member may additionally comprise a marking that indicates the location of the joint line of the packing material to the user. This marking may be applied to the flange using means known in the art including, but not limited to, pad printing, injection molding, inscribing, label application, combinations thereof, and the like. Alternatively, the marking may be applied to other parts of the device of the invention. The marking may be general (e.g. the joint of the packing material is located within an arc of 0°-180° or 180°-360°) or specific (e.g. the joint of the packing material is located within an arc of 5° increment such as 0°-5°, 180°-185°, and the like). It should be clear to one of skill in the art that the precision of the marking may be as fine or coarse as needed for a particular application.

An exemplary method of use for this embodiment of the invention is in the treatment of epistaxis. The device of the invention is inserted into the target nostril until the flange contacts the alar rim of the patient is and rotated until the marking indicating the location of joint of the packing material is facing away from the nasal septum. An air-filled syringe is connected to the luer activated valve and air is injected into the infusion line to expand the expandable member away from the elongate member. At a sufficient pressure, the expandable member breaks the packing material along the joint and presses the packing material against the nasal septum to halt the bleeding. The syringe is decoupled from the luer activated valve without deflating the expandable member to enable the device to continue to apply pressure to the source of the bleeding. After a sufficient time has passed (per standard of care at the treatment facility), the syringe is re-attached to the luer activated valve, the expandable member is deflated, and the device is removed from the patient.

While this embodiment of the invention has described an expandable member comprising a flexible and/or elastic material that is activated by an increase in the internal pressure of the device, it should be clear to one of skill in the art that other means of achieving and expandable member are possible. For example, the expandable member may be a stent or stent-like structure that undergoes an increase in dimension through the use of a pull-wire mechanism.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A through 1H show examples of an elongate member and the different numbers and geometries of lumens that may be incorporated in the invention.

FIGS. 2A through 2F show different embodiments of the flange of the elongate member.

FIGS. 3A and 3B illustrate exemplary embodiments of lumens and channels in the elongate member.

FIGS. 4A through 4D illustrate exemplary embodiments of the plunger and the relationship between the plunger and the elongate member.

FIGS. 5A and 5B illustrate two exemplary embodiments of the fluid delivery member.

FIGS. 6A and 6B illustrate an embodiment of the invention for treatment of epistaxis.

FIG. 7 depicts a method of using an embodiment of the invention to treat epistaxis.

FIGS. 8A through 8C depict an embodiment of the invention for delivering a payload into the nasal cavity or paranasal sinuses.

FIGS. 9A and 9B depict an alternative embodiment of the invention for delivering a packing material into the nasal cavity or paranasal sinuses.

FIG. 10 depicts another alternative embodiment of the invention for delivering a packing material into the nasal cavity or paranasal sinuses.

FIGS. 11A through 11C depict top, side, and cross-sectional views of an embodiment of the invention for delivery a packing material into the nasal cavity.

FIGS. 12A and 12B depict side and cross-sectional views of the embodiment of the invention of FIG. 11 with the shuttle retracted.

FIGS. 13A and 13B depict top and side views of an embodiment of the invention for delivery a packing material into the nasal cavity, along with cross-sectional views of proximal portions of the embodiment of the invention.

FIGS. 14A and 14B depict side cross-sectional views of the embodiment of the invention of FIG. 13 with the actuator in the neutral and depressed states.

FIGS. 15A through 15C depict an alternative embodiment of a means of releasing a packing material into the nasal cavity.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a compound” includes a plurality of such compounds and reference to “the polymer” includes reference to one or more polymer and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

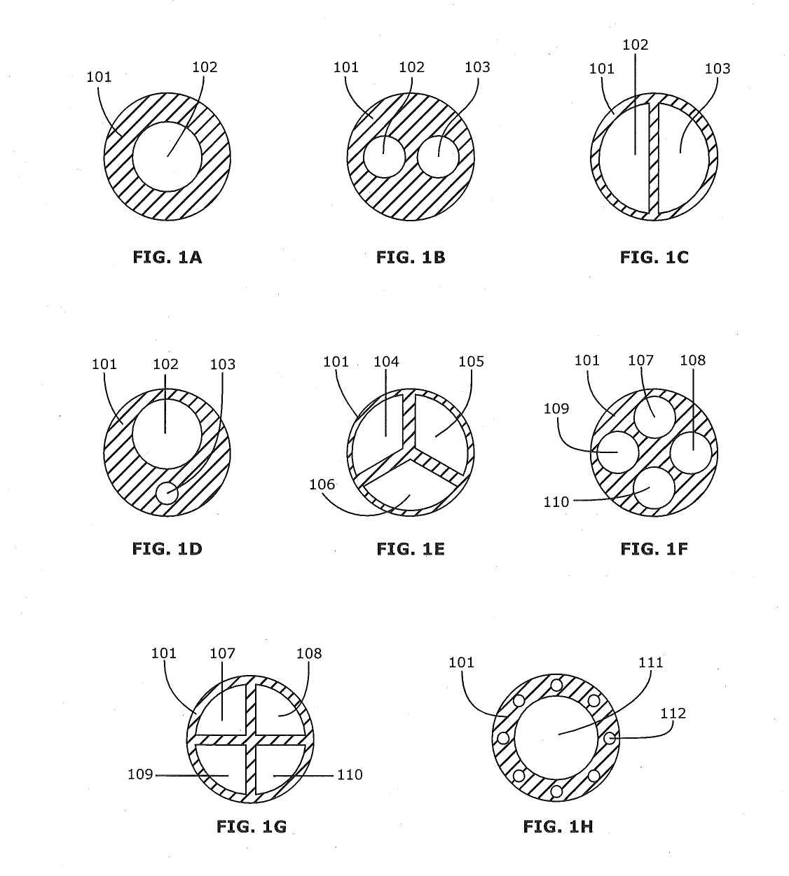

Examples of the elongate member 101 with varying numbers and geometries of the lumens disclosed in this invention are illustrated in cross-section views shown in FIGS. 1A through 1H. FIG. 1A is an illustration of a single lumen 102 in an elongate member 101. While lumen 102 is shown a concentrically aligned with elongate member 101, it should be clear to those of skill in the art that lumen 102 may possess any size and/or location within the boundary established by the outer diameter of elongate member 101. FIG. 1B shows an elongate member 101 with two lumens, 102 and 103. While lumens 102 and 103 are shown as being circular in cross-section, identical in diameter, and symmetric about the vertical and horizontal centerlines of the cross-section of elongate member 101, it should be obvious to one of skill in the art that other arrangements of lumens 102 and 103 are possible. For instance, FIG. 1C shows lumens 102 and 103 as having identical “D” shapes and maintaining symmetry about the vertical and horizontal centerlines of the cross-section of elongate member 101. Another example is shown in FIG. 1D, wherein lumen 102 is much larger than lumen 103, and the pair of lumens is symmetric about the vertical centerline of the cross-section of elongate member 101 and non-symmetric about the horizontal centerline of the cross-section of elongate member 101. FIGS. 1E through 1F show examples of an elongate member 101 with three (3) lumens 104, 105, 106 as shown in FIG. 1E, four lumens 107, 108, 109, and 110 as shown in FIGS. 1F and 1G, and nine (9) total lumens with eight (8) lumens 112 and one (1) lumen 111 as shown in FIG. 1H. Referring to FIG. 1E, while lumens 104, 105, and 106 are shown as having an identical wedge shape and an even radial orientation about the center point of the cross section of elongate member 101, it should be clear that other sizes, shapes, and relative spatial arrangements of lumens 104, 105, and 106 are contemplated. FIG. 1F is one example of an elongate member 101 where lumens 107, 108, 109, and 110 are shown as circular in cross-section, identical in size, and symmetric about the horizontal and vertical centerlines of the cross-section of elongate member 101. FIG. 1G is an alternative example of an elongate member 101 showing identical and symmetric lumens 107, 108, 109 and 110. FIG. 1H shows an elongate member 101 comprising one large lumen 111 and eight identical smaller lumens 112. The lumens 112 are located in the wall of elongate member 101 and are located at angles of 0°, 45°, 90°, 135°, 180°, 225°, 270°, and 315° from the intersection of the horizontal and vertical centerlines of the cross-section of elongate member 101. The examples shown in these figures are not an exhaustive list of the configurations of an elongate member and one or more lumens, and it should be clear that many such configurations are possible and are contemplated by this invention.

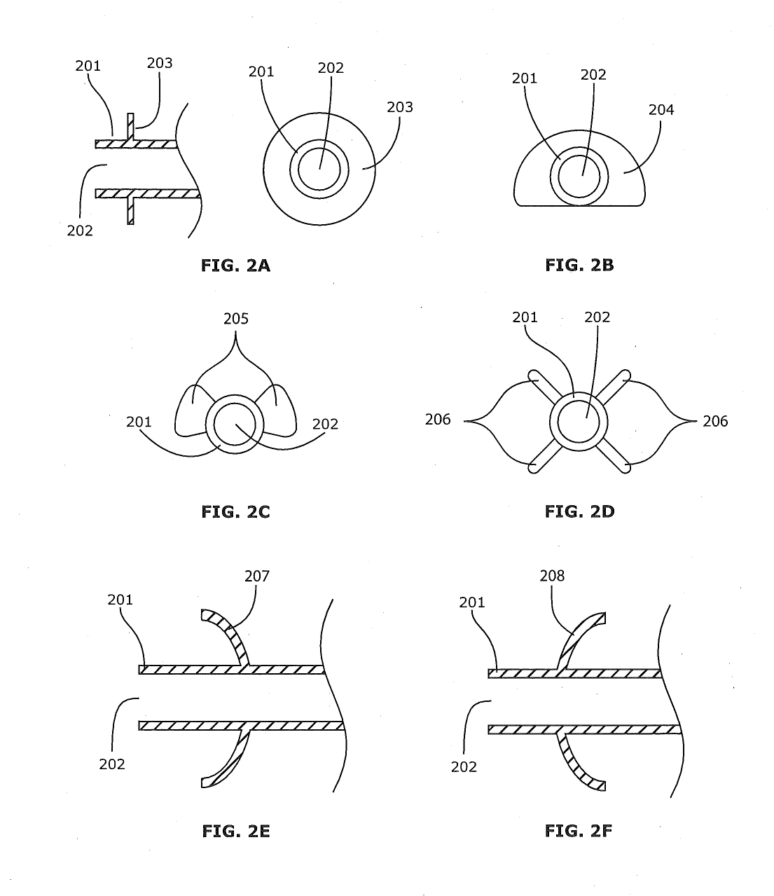

Examples of the feature component of the elongate member are illustrated in FIGS. 2A through 2F. FIG. 2A shows a transverse cross section and a front view of an elongate member 201 comprising a lumen 202 and a feature 203 shaped as a circular flange. In this example, the feature 203 and lumen 202 are concentrically aligned with the elongate member 201. FIG. 2B is an example of a non-circular feature 204 that is not concentrically aligned with elongate member 201 and lumen 202, shown in a front view. This design may be preferred in applications where a centered feature (as shown in FIG. 2A) would not be able to access the desired anatomy, would have more difficulty accessing the desired anatomy, or would be uncomfortable or harmful to the patient. One such application would be inserting the delivery device into a nostril and using the alar rim and/or columella as an anchor or stabilization point; the flat segment of the asymmetric feature 204 could rest comfortably against the nasal sill while the rounded segments of feature 204 could contact and stabilize the device against the alar rim and/or columella. FIG. 2C is a front view of an elongate member 201 comprising a lumen 202 and a feature 205, wherein feature 205 comprises two wings or wedges. A feature of this shape may be beneficial in locating the delivery device at a particular anatomical feature, as the number, size, shape, and arrangement of the wings or wedges may be chosen to match the anatomical target of interest. FIG. 2D is a front view of an elongate member 201 comprising a lumen 202 and a feature 206, wherein feature 206 comprises four narrow extensions arranged along lines aligned at 45°, 135°, 225°, and 315° with respect to the intersection of the horizontal and vertical centerlines of elongate member 201. It should be clear to one skilled in the art that the number, shape, length, and radial location of the extensions comprising feature 206 may take any one of a set of myriad permutations. For example, feature 206 may alternatively comprise four extensions that are joined to elongate member 201 in the same location as shown in FIG. 2D, have the same length and thickness as shown in FIG. 2D, but differ in that each of the four extensions assumes a clockwise spiral arc as the distance from elongate member 201 increases. Similarly, the same extensions could arc counterclockwise as the distance from elongate member 201 increases, or assume any other shape without changing either the length or thickness of the individual extensions. FIGS. 2E and 2F are transverse cross sections of an elongate member 201 comprising a lumen 202 and either a feature 207 that arcs distally or a feature 208 that arcs proximally as the distance from elongate member 201 increases. While the examples shown in FIGS. 2E and 2F illustrate relatively simple geometries for features 207 and 208, it should be clear to one of skill in the art that any shape may be possible.

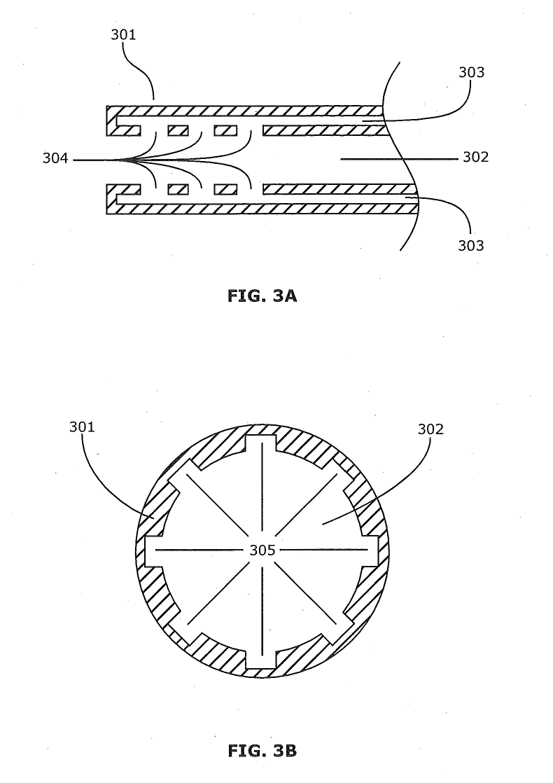

FIG. 3A provides an illustration of the distal end of an elongate member 301 comprising a central lumen 302, peripheral lumens 303, and multiple holes or apertures 304. It should be noted that the number and position of holes or apertures 304 can vary, and that all such permutations are contemplated by this disclosure. Furthermore, additional holes or apertures (not shown) may be located at the distal end of elongate member 301 such that any material flowing through peripheral lumens 303 may exist peripheral lumens 303 in the distal direction. In this example, holes or apertures 304 may or may not present in elongate member 301. FIG. 3B shows a cross-sectional view of an elongate member 301 comprising a central lumen 302 and multiple channels 305. Again, the number, location, size and or depth of channels 305 may vary from the exemplary illustration shown in FIG. 3B, and permutations of such are contemplated in this disclosure. It should be clear to one of skill in the art that an elongate member of the invention may comprise any or all of the lumens, channels, features, holes, or apertures shown in FIGS. 1 through 3.

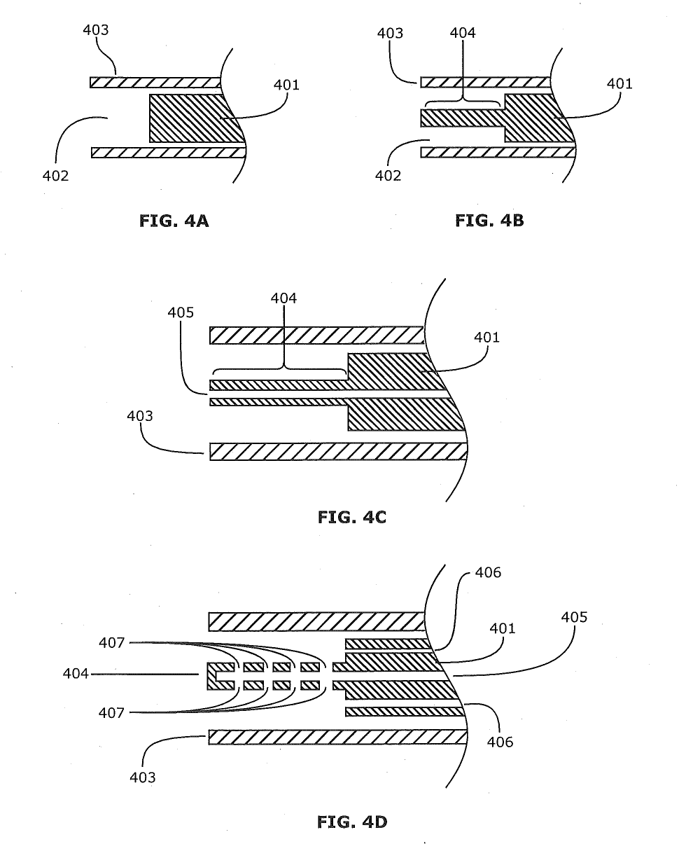

FIG. 4A is a transverse cross section showing plunger 401 slidably disposed within the lumen 402 of elongate member 403. FIG. 4B is a transverse cross section showing plunger 401 slidably disposed within the lumen 402 of elongate member 403. Plunger 401 further comprises a cylindrical tube or an extrusion 404 extending from the distal face of plunger 401. The length and outer dimension (e.g. the outer diameter in the case of a cylindrical extrusion) of extrusion 404 can cover a wide range of measurements, and can preferably be chosen to accommodate inclusion of a nasal packing material, a stent, or other material (not shown) in the space between elongate member 403 and extrusion 404. FIG. 4C is a transverse cross section of a plunger 401 comprising an extrusion 404 and a lumen 405 slidably disposed within elongate member 403. While lumen 405 is depicted as coaxially disposed within plunger 401, it should be clear to one skilled in the art that additional lumens located at any position within plunger 401 are contemplated. Furthermore, the inclusion of a lumen 405 within plunger 401 is not dependent on the presence of extrusion 404; that is, lumen 405 may be present in plungers 401 of the type shown in FIG. 4A. FIG. 4D is a transverse cross section of a plunger 401 comprising a central lumen 405, peripheral lumens 406, extrusion 404, and exit ports or apertures 407 slidably disposed within the lumen of elongate member 403. Alternatively (not shown), central lumen 405 may extend through the distal tip of extrusion 404.

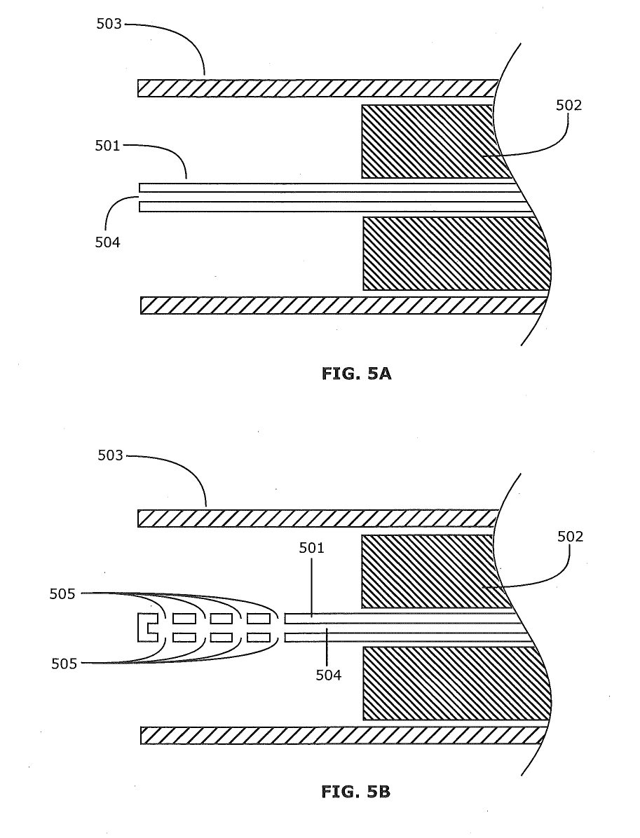

FIG. 5A is a transverse cross section of a fluid delivery member 501 coaxially disposed within a lumen of plunger 502 and elongate member 503. Fluid delivery member 501 further comprises a lumen 504, and is positioned such that the distal end of fluid delivery member 501 is aligned with the distal end of elongate member 503. Alternatively (not shown), the distal end of fluid delivery member may be offset either proximally or distally from the distal end of elongate member 503. Furthermore, fluid delivery member 501 may be fixed in translation with respect to elongate member 503 and/or plunger 502. For example, an embodiment of the invention that comprises a movable elongate member 503 and a fixed plunger 502 may further comprise a fixed fluid delivery member. In another example, an embodiment of the invention may comprise a fixed plunger 502, with both elongate member 503 and fluid delivery member 501 free to move. In this example, a payload (not shown) may be loaded into the space bounded by the inner surface of elongate member 503, the outer surface of fluid delivery member 501, and the distal end of plunger 502. Once the device is positioned to deliver the payload to the desired target anatomy, the following general sequence of steps occurs: (1) elongate member 503 is retracted proximally with respect to the fixed plunger 502 to expose the payload, (2) fluid delivery member 501 is retracted proximally with respect to fixed plunger 502 to release the payload (not shown), and (3) the delivery device of the invention is removed from the target anatomy. Fluid may be delivered through fluid delivery member 501 at any point in this general sequence. Furthermore, if elongate member 503 or fixed plunger 502 comprise additional lumens as described in FIG. 3A, 3B, or 4D, fluid may be delivered through these additional lumens at any point in the general sequence previously described or as desired by a user. Delivery of fluid through multiple independent lumens may be performed in a synchronous or asynchronous manner Examples include, but are not limited to all lumens delivering the same type of fluid at the same time, each lumen delivering one of a selection of fluids at an independent time, each lumen delivering an identical fluid at a different time, each lumen delivering a unique fluid at the same time, combinations thereof, and the like. Different volumes of fluid may be delivered through the different lumens, and the duration of the delivery times may vary based on factors including, but not limited to the viscosity of the fluid, the size and shape of the lumen, the delivery of a desired dose of a medication or therapeutic agent (in the case of fluids that comprise a therapeutic agent), the volume of fluid needed to hydrate an absorbent payload, combinations thereof, and the like.

In yet another example, a delivery device of the invention may comprise a fixed elongate member 503, a fixed fluid delivery member 501, and a movable plunger 502. A payload may be loaded into the space bounded by the inner surface of elongate member 503, the outer surface of fluid delivery member 501, and the distal end of plunger 502. Once the device is positioned to deliver the payload to the desired target anatomy, the following general sequence of steps occurs: 1) the plunger 502 is advanced distally with respect to the elongate member 503 and the fluid delivery member 501 to push the payload out of the lumen of elongate member 503, and 2) the delivery device of the invention is removed from the target anatomy. As with the prior example, fluid may be delivered through lumen 504 of fluid delivery member 501 at any point in this general sequence and the use of elongate members and/or plungers comprising addition lumens for fluid delivery as previously described is contemplated by this invention.

FIG. 5B shows the elongate member 503, plunger 502, fluid delivery member 501 and fluid delivery member lumen 504 of FIG. 5A wherein the fluid delivery member 501 further comprises at least one hole or aperture 505. The at least one hole or aperture 505 extends from fluid delivery member lumen 504 to the surface of fluid delivery member 501. It should be clear that any number and/or arrangement of holes or apertures 505 in fluid delivery member 501 are contemplated in this invention, and that the size, shape, orientation, and location of the at least one hole or aperture 505 may be altered or modified to suit a particular application.

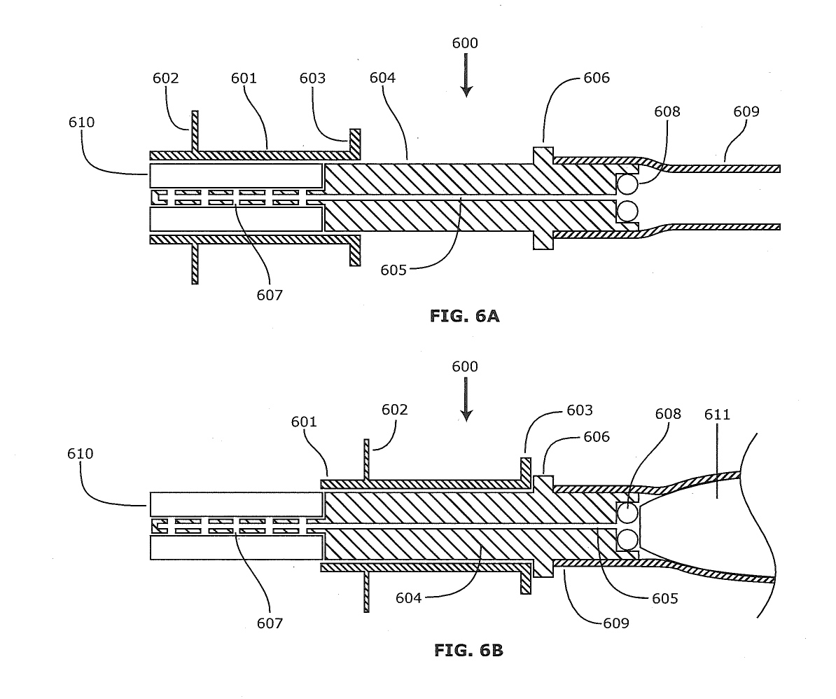

FIG. 6A depicts a transverse cross section of an embodiment of the invention 600 suited for the treatment of epistaxis comprising an elongate member 601, a plunger 604, an o-ring 608, elastic connector 609, and a nasal packing material, stent or other material 610. Elongate member further comprises a distal flange 602 and an optional proximal flange 603. While distal flange 602 and proximal flange 603 are depicted as laterally extending symmetric features in transverse cross section, it should be clear to one of skill in the art that these features may take any geometry. Plunger 604 further comprises a lumen 605, a stopper 606, an extrusion extended from the distal face of the plunger body, and a series of holes or apertures 607. Plunger 604 is slidably disposed within and concentrically aligned with elongate member 601. Proximal flange 603 and stopper 606 are sized such that stopper 606 can not translate distally past the proximal face of proximal flange 603 of elongate member 601, or conversely, such that proximal flange 603 can not translate proximally past the distal face of stopper 606. Nasal packing material 610 is positioned within the space defined by the inner surface of elongate member 601, the outer surface of the plunger extrusion, and the distal face of plunger 604. O-ring 608 is positioned about plunger lumen 605 at the proximal end of plunger lumen 605. Elastic connector 609 may be fabricated from an elastic material such as silicone rubber, and stretched over the proximal portion of plunger 604. Furthermore, the proximal portion of elastic connector 609 may be sufficiently elastic to fit over, secure and seal against the nozzle 611 of a fluid reservoir (e.g. a squeeze bottle) (not shown). Nasal packing material 610 may be any of those previously described herein, but may preferably be a xerogel formulated as disclosed in co-pending U.S. patent application 61/259,564.

FIG. 6B depicts the embodiment of the invention 600 joined to a fluid reservoir nozzle 611 wherein the plunger 604 is fully inserted into elongate member 601 such that the proximal flange 603 of elongate member 601 abuts stopper 606, exposing nasal packing material 610 to the environment in the immediate vicinity of the target anatomy. The advancement of plunger 604 with respect to elongate member 601 may be achieved by stabilizing the distal feature 602 against a desired anatomical feature, such as the alar rim and/or columella. The nozzle 611 of the fluid reservoir is joined to the embodiment of the invention 600 by the proximal portion of elastic connector 609. Furthermore, nozzle 611 abuts and seals against o-ring 608, creating a flow path from nozzle 611, through plunger lumen 605, and out of the series of holes or apertures 607. The fluid contained within the reservoir may be any of those previously described herein, but may preferably be a solution of 0.5% oxymetazoline in buffered saline, commercially available under the trade name of Afrin® (Schering-Plough HealthCare Products, Inc.).

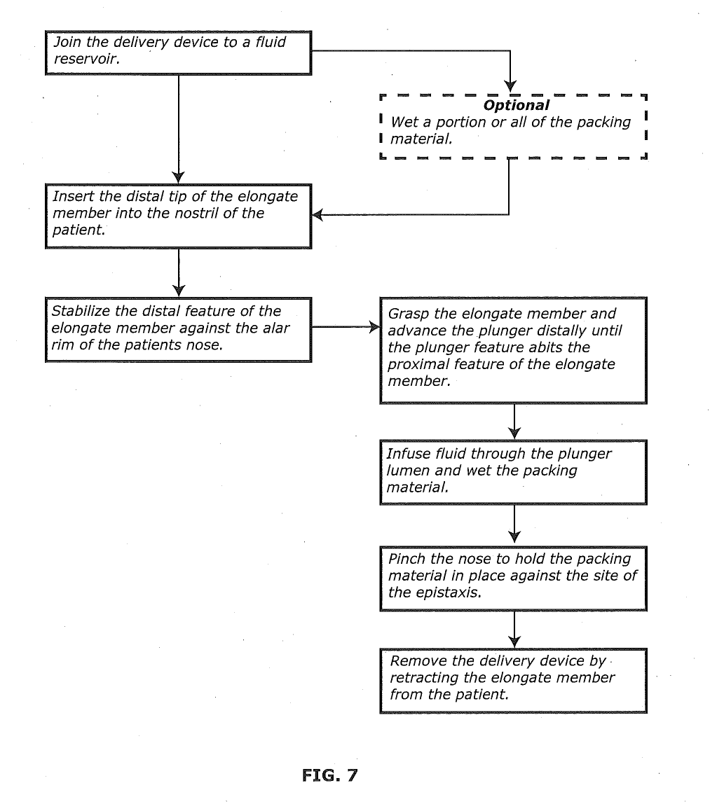

One method of using embodiment of the invention 600 (as detailed in FIGS. 6A and 6B) to treat epistaxis is shown in the flowchart depicted in FIG. 7. In a first step, the delivery device 600 is joined to a fluid reservoir. As an exemplary embodiment the connector 609 is shown previously as a component that can be stretched over and secured about the outside surface of the nozzle 611 of a typical squeeze bottle reservoir (e.g. a typically used Afrin bottle), it is clear that the delivery device could be connected to the desired fluid reservoir using any number of joining modalities (e.g. luer locks, slip luers or any modality that adequately joins and secures the delivery device with the reservoir). The nasal packing material 610 disposed in the distal portion of delivery device 600 may optionally be wetted prior to enacting the second step of the method. In a second step, the distal portion of elongate member 601 is inserted into the nostril of the patient. The delivery device 600 is then advanced into the nasal cavity until the distal flange 602 contacts the alar rim and/or columella (not shown) of the patient in a third step. In a fourth step, the body of elongate member 601 is grasped and plunger 604 is advanced distally until stopper 606 contacts proximal flange 603 of elongate member 601. Fluid is then infused into the nasal packing material 610 through plunger lumen 605 and holes or apertures 607 in a fifth step. In a sixth step, the patient or physician or caregiver then applies external pressure or digital compression to the nose (e.g. pinches the nose) to hold and secure the nasal packing material 610 against the source of the bleeding or epistaxis. As a final step, the delivery device 600 is withdrawn while external pressure or digital compression is maintained. It should be obvious that (if desired) the digital compression could be released prior to device removal in the final step of the method described herein as well. This method is shown exemplary embodiment only and it should be obvious to those skilled in the art that the individual steps of the method may be permuted as desired to gain the desired clinical effect.

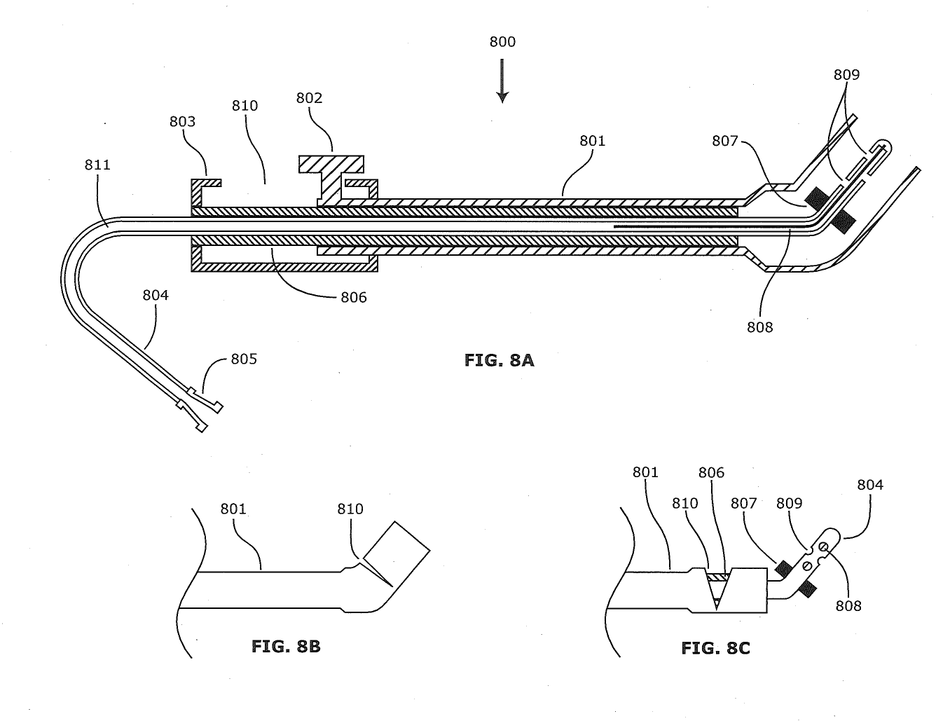

FIG. 8A is a transverse cross-section of an embodiment of the delivery device 800 that may be useful in deploying a nasal pack, stent or other material to the nasal cavity and/or paranasal sinuses. Embodiment 800 comprises an elongate member 801, a handle 803, an infusion tube 804, an infusion port 805, plunger 806, a backstop 807, and a support member 808. Elongate member 801 further comprises a slide button 802 located in the proximal portion of elongate member 801. Handle 803 further comprises a channel 810. Infusion tube 804 further comprises at least one infusion lumen 811 and exit ports or apertures 809. Exit ports 809 are preferentially located in the distal portion of infusion tube 804, and are even more preferentially located distal to backstop 807 and all along the length of the space where the payload (not shown) would be positioned at the distal portion of delivery device 800. Elongate member 801 is slidably disposed within handle 803 and oriented such that slide button 802 can move in the proximal or distal direction within channel 810 of handle 803. Plunger 806 is disposed within and coaxially disposed within elongate member 801. The distal end of plunger 806 is joined to handle 803 using techniques known in the art including, but not limited to adhesive bonding, welding, ultrasonic welding, overmolding, threading and tapping, mechanical fixation, friction or interference fits, combinations thereof, and the like such that elongate member 801 can translate in the proximal and distal directions with respect to plunger 806. Plunger 806 is preferably stiffer than elongate member 801. Infusion tube 804 is coaxially disposed within plunger 806 and may optionally be joined to plunger 806 using techniques known to the art including, but not limited to adhesive bonding, welding, ultrasonic welding, overmolding, threading and tapping, mechanical fixation, friction or interference fits, combinations thereof, and the like. The proximal end of infusion tube 804 is joined to infusion port 805 using techniques known to the art including, but not limited to adhesive bonding, welding, ultrasonic welding, overmolding, threading and tapping, mechanical fixation, friction or interference fits, combinations thereof, and the like. Infusion port 805 may be of any configuration or shape that enables a connection between infusion tube 804 and a source of fluid, but preferably is a luer fitting, such as a male or female luer lock or male or female slip luer commonly used in the art. Alternatively, the infusion port may comprise an elastic connector as described previously and serve as a means to secure the delivery device 800 to the desired fluid reservoir. The distal portion of infusion member 804 comprises exit ports 809. The distal end of infusion member 804 may be formed into an atraumatic shape such as a ball, hemisphere, tapered tip, and the like. Support member 808 is disposed within infusion lumen 811 of infusion member 804. The distal end of support member 808 is joined to the distal end of infusion member 804 using techniques known to the art including, but not limited to adhesive bonding, welding, ultrasonic welding, overmolding, threading and tapping, mechanical fixation, friction or interference fits, combinations thereof, and the like. Support member 808 may extend a distance proximally within infusion member 804, and may preferentially be sized such that fluid infused through infusion lumen 811 can pass around support member 808 and out of exit ports 809. Alternatively, the support member 808 may extend proximally and be secured within infusion lumen 811 of infusion member 804 using bonding methods well known in the art. Support member 808 may be adjusted to any angle desired by the surgeon, but in one preferred embodiment, is adjusted to approximately 70° (as measured from the long axis of the plunger 806) to improve the capability to access the frontal sinus or frontal sinus recess or the frontal sinus ostium. Other angles and orientations suited to accessing the maxillary sinus or maxillary sinus ostium, an ethmoid sinus or an ethmoid sinus ostium (either a natural ostium or a surgical antrostomy) or ethmoid infindibulum, or the sphenoid sinus or sphenoid sinus ostium are contemplated. It is obvious to those skilled in the art that support member 808 may be substituted with an infusion tube 804 that is constructed from a material is itself rigid and malleable eliminating the need for support member 808. It is contemplated that the rigid and malleable infusion tube 804 or support member 808 may be constructed from materials including, but not limited to stainless steel, titanium, nickel titanium, alloys thereof and numerous other metallic materials well known in the art. Backstop 807 is coaxially disposed about and joined to infusion tube 804 using techniques known to the art including, but not limited to adhesive bonding, welding, ultrasonic welding, overmolding, threading and tapping, mechanical fixation, friction or interference fits, combinations thereof, and the like. Backstop 807 may be preferentially sized to prevent the payload (not shown) from moving distally when elongate member 801 translates in the distal direction, and may be preferentially located such that the distal face of backstop 807 is offset from the distal end of the infusion tube 804 a distance equal to the length of the payload. For example, a payload that is 4.0 cm in length may require the backstop 807 to be positioned such that the distal face of backstop 807 is 4.0 cm from the distal end of infusion tube 804.

FIGS. 8B and 8C illustrate the action of the elongate member 801 when slide button 802 is moved from the distal to proximal positions within channel 810 of handle 803 (as shown in FIG. 8A). As seen in FIG. 8B, elongate member 801 further comprises a slit 812 located in the distal portion of elongate member 801. Slit 812 may preferably be a v-cut that extends over 50% of the diameter of elongate member 801. Slit 812 may also comprise other geometries known to the art, including but not limited to angular notches, arcs, channels, combinations thereof, and the like. Retraction of elongate member 801 in the proximal direction pulls the distal portion of elongate member 801 over plunger 806, slit 812 and straightening the distal portion of elongate member 801 to expose the payload (not shown) and the distal portion of infusion tube 804, as shown in FIG. 8C.

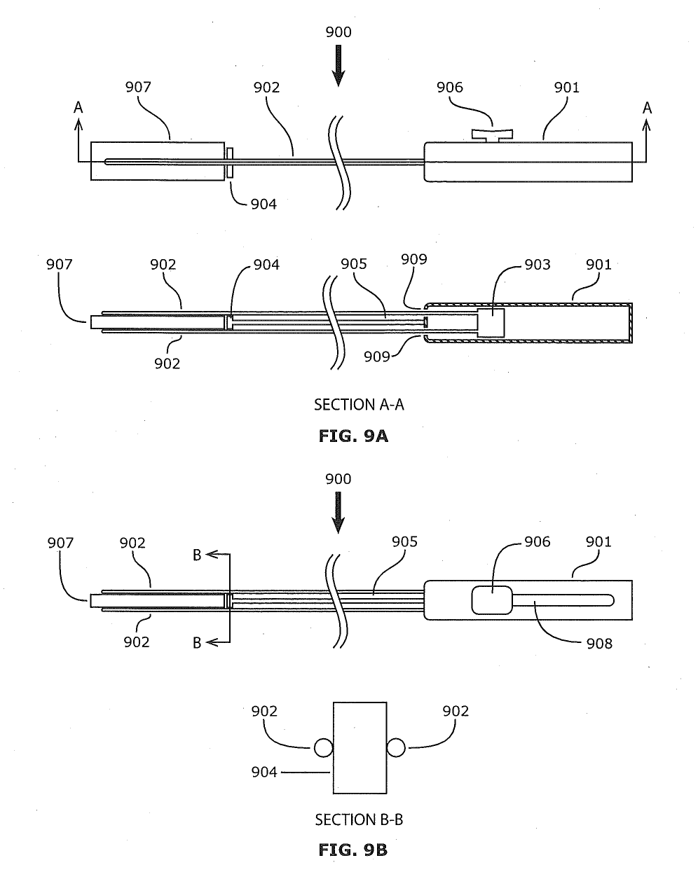

FIG. 9A is a side view of an embodiment of the invention 900 and a cross-sectional view of embodiment 900 along the section line A-A. Embodiment 900 comprises a handle 901, tines 902, common elongate member 903, footplate 904, additional elongate member 905, and actuator 906. Packing material 907 is shown for reference. FIG. 9B is a top view of embodiment 900 depicting slot 908 in handle 901 and a cross-sectional view of footplate 904 and tines 902 along section line B-B. In both figures, the distal portion of tines 902 grasp packing material 907 and the proximal portion of tines 902 merge into common elongate member 903. Common elongate member 903 is slidably disposed within handle 901 and connected to actuator 906. Handle 901 further comprises two thru holes 909 that are sized to accept tines 902 with sufficient clearance to allow tines 902 to translate freely through the distal face of handle 901. Actuator 906 is slidably disposed within slot 908 such that moving actuator 906 in the proximal direction moves the distal ends of tines 902 in the proximal direction and moving actuator 906 in the distal direction moves the distal ends of tines 902 in the distal direction. The footplate 904 is positioned proximal to nasal packing 907 and is fixed in position relative to tines 902 by additional elongate member 905. Additional elongate member 905 is relatively rigid, and the proximal end of elongate member 905 is joined to the distal face of handle 902. Footplate 904 and additional elongate member 905 act to prevent the proximal movement of nasal packing 907 when actuator 906 is translated in the proximal direction in the event that tines 902 provide sufficient inward force to hold on to packing material 907 during proximal translation. The proximal face of packing material 907 contacts the distal face of footplate 904 as tines 902 are retracted proximally, preventing further proximal motion of packing material 907 and enabling tines 902 to slide clear of nasal packing 907. The width of footplate 904 (as shown in section B-B) may be line to line with the inner surfaces of tines 902. Alternatively (not shown), the width of footplate 904 may extend such that tines 902 are partially or completely enclosed within the distal face of footplate 904. In these cases, cutouts or thru holes of sufficient size, location, and clearance to allow tines 902 to translate freely in the proximal and distal directions are incorporated into footplate 904. As shown in FIG. 9A, the height of footplate 904 is preferably large enough to present a sufficiently large obstacle to the proximal movement of packing material 907. However, the overall size of footplate 907 should preferably not exceed the cross sectional area of packing material 907 to enable adequate maneuverability of the device in the nasal cavity. Packing material 907 may be any of those previously described herein, but may preferably be a xerogel formulated as disclosed in co-pending U.S. patent application 61/259,564, the disclosure of which is incorporate by reference in its entirety.

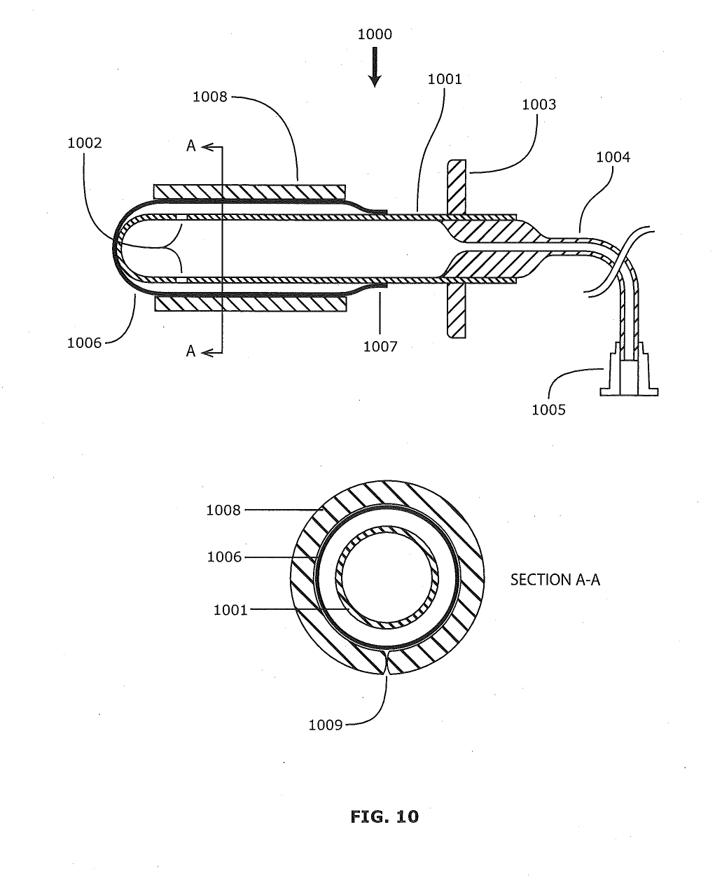

FIG. 10 depicts an embodiment of the invention 1000 in a top cross-sectional view and a transverse cross section through section A-A. Embodiment 1000 comprises an elongate member 1001, flange 1003, extension line 1004, connection port 1005, expandable member 1006, and packing material 1008. Elongate member 1001 further comprises injection ports 1002. Flange 1003 is joined to elongate member 1001. The distal end of extension line 1004 is joined to the proximal end of elongate member 1001 and the proximal end of extension line 1004 is joined to the distal end of connection port 1005. Expandable member 1006 is disposed about the distal portion of elongate member 1001 and sealed to elongate member 1001 at joint 1007. Packing material 1008 is disposed about expandable member 1006. In this embodiment, packing material 1008 further comprises joint line 1009. Packing material 1008 may be any of those previously described herein, but may preferably be a xerogel formulated as disclosed in co-pending U.S. patent application 61/259,564, the disclosure of which is incorporate by reference in its entirety.

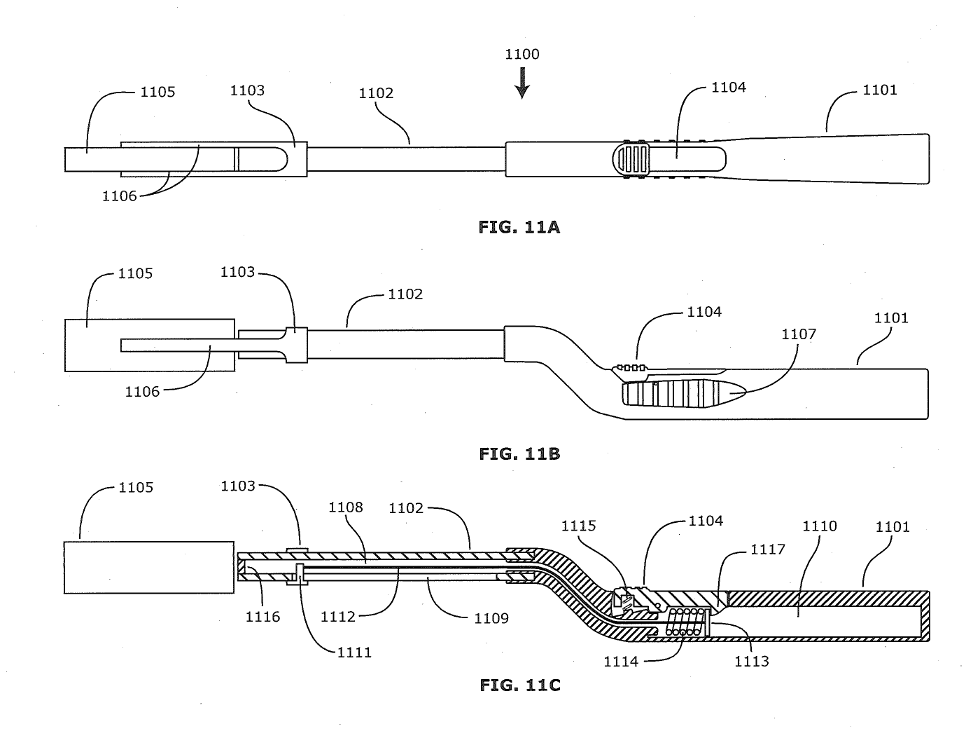

FIGS. 11A and 11B depicts top and side views, respectively, of an embodiment of the invention 1100. FIG. 11C depicts a cross-sectional side view of the embodiment of the invention. Embodiment 1100 comprises a handle 1101, an elongate member 1102, a shuttle 1103, an actuator 1104, and a packing material 1105. Packing material 1105 may be any of those previously described herein, but may preferably be a xerogel formulated as disclosed in co-pending U.S. patent application 61/259,564, the disclosure of which is incorporate by reference in its entirety. Shuttle 1103 further comprises tines 1106 and a post 1111. In this embodiment there are two tines 1106, however it should be clear to one of skill in the art that shuttle 1103 may comprise any number of tines or means of holding packing material 1105. Handle 1101 further comprises a ridged and/or contoured section 1107. Elongate member 1102 further comprises at least one lumen 1108, a channel 1109 extending a portion of the length of elongate member 1102 and, in this example, positioned on the bottom elongate member 1102, and a cap 1116 joined to the distal end of elongate member 1102. It should be clear to one of skill in the art that the shape, length, orientation with respect to the elongate member 1102, and the width of channel 1109 may be varied to accommodate differing specific designs of the embodiment of the invention. Cap 1116 is joined to the distal end of elongate member 1102 using means known to the art, including but not limited to adhesive bonding, welding, ultrasonic welding, overmolding, threading and tapping, mechanical fixation, friction or interference fits, combinations thereof, and the like. Alternatively, cap 1116 may be an integral part of the elongate member 1102 that is formed or cut using processes known in the art such as stamping, laser cutting and the like and folded in to the lumen opening of the elongate member 1102. The proximal end of elongate member 1102 is joined to the distal end of handle 1101 using means known to the art, including but not limited to adhesive bonding, welding, ultrasonic welding, overmolding, threading and tapping, mechanical fixation, friction or interference fits, combinations thereof, and the like. Handle 1101 further comprises an internal space 1110 that is in communication with lumen 1108 of elongate member 1102. Link member 1112 is slidably disposed within lumen 1108 of elongate member 1102 and extends into the internal space 1110 of handle 1101. The distal end of link member 1112 is joined to post 1111 using means known to the art, including but not limited to adhesive bonding, welding, ultrasonic welding, overmolding, threading and tapping, mechanical fixation, friction or interference fits, combinations thereof, and the like. Link member 1112 is flexible to enable translation of link member 1112 through the S-shaped distal portion of handle 1101. The proximal end of link member 1112 is joined to disk 1113 using means known to the art, including but not limited to adhesive bonding, welding, ultrasonic welding, overmolding, threading and tapping, mechanical fixation, friction or interference fits, combinations thereof, and the like. Spring 1114 is disposed within internal space 1110 of handle 1101 and about the proximal portion of link member 1112. Spring 1114 is compressed such that the proximal end of spring 1114 abuts the distal face of disk 1113. Spring 1114 is prevented from expanding to its unloaded length by the interaction between the proximal latch 1117 of actuator 1104 and the proximal face of disk 1113. Actuator 1104 is biased into a position such that proximal latch 1117 restricts the proximal motion of disk 1113.

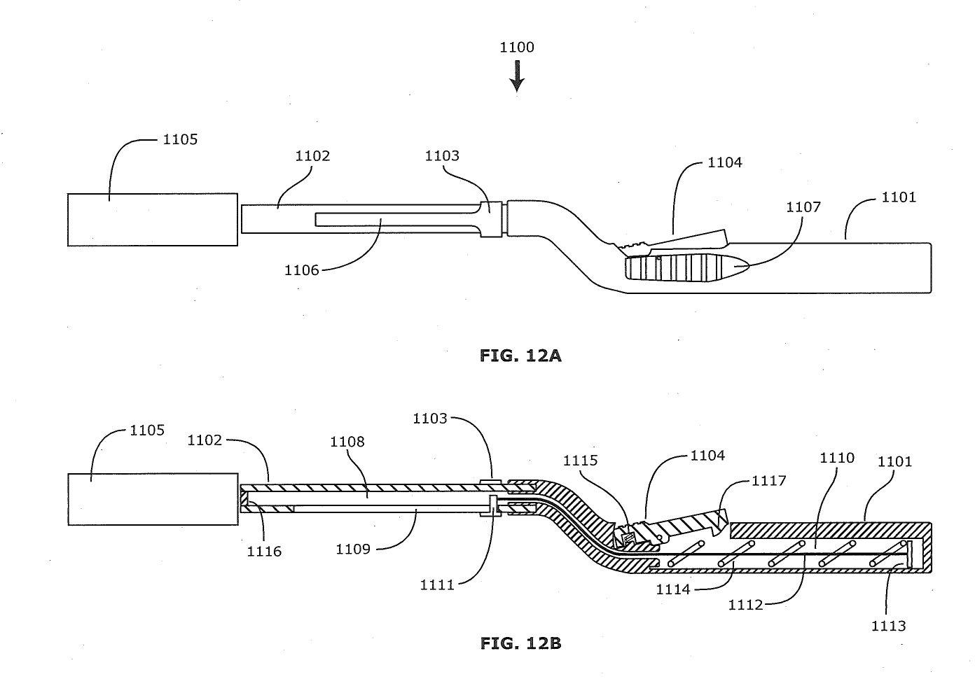

FIGS. 12A and 12B show side and cross-sectional views of the embodiment of the invention 1100 after the distal portion of actuator 1104 has been depressed. The action of depressing the distal portion of actuator 1104 with sufficient force to overcome the resisting force of spring 1115 raises the proximal latch 1117 of actuator 1104. When the proximal latch 1117 is moved out of contact with the proximal face of disk 1113, spring 1114 expands to its unloaded position (i.e. the proximal end of spring 1114 translates proximally within internal space 1110) and drives disk 1113 to the proximal portion of internal space 1110. The proximal motion of disk 1113 is transmitted through link member 1112 to post 1111, and to shuttle 1103. The proximal motion of shuttle 1103 relative to elongate member 1102 pulls tines 1106 and packing material 1105 in the proximal direction. The proximal end of packing material 1105 contacts the distal face of cap 1116 and the distal end of elongate member 1102, halting the proximal motion of packing material 1105. Tines 1106 continue to translate in the proximal direction, releasing packing material 1105 from device 1100. While the embodiment of the invention shown in FIGS. 11 and 12 is intended for single use, it should be clear to one of skill in the art that a re-usable embodiment may be fabricated by providing a means to push disk 1113 in the distal direction such that actuator 1104 may be re-set in its original position. In such an embodiment, link member 1112 may preferably be fabricated from a material, or in such a manner, that it is both flexible and can support compressive loads such that distal translation of disk 1113 results in distal translation of shuttle 1103. For example, stainless steel formed into a coiled coil (such as found in cardiovascular guidewires) or a nickel-titanium alloy may preferably be chosen. Re-useable alternatives to the aforementioned embodiment of the invention may preferably be manufactured from materials that are resistant to multiple rounds of sterilization (e.g. steam autoclaving). Alternatively (not shown), apparatus 1100 can be configured such that the spring 1114 is located at the proximal side of disk 1113 wherein its proximal end is affixed or anchored to the proximal end of handle 1101. Load to the spring 1114 is applied by stretching the distal end until disk 1113 engages to latch 1117 of actuator 1104. Other types of spring 1114 known in the art may also be used such as constant force spring, elastic rubber and the like.

Apparatus 1100 may also have additional features such as an adjustable elongate member 1102 wherein the elongate member 1102 can be rotated along its longitudinal central axis (not shown). This feature allows adjustment of the packing material 1105 to vary its position relative to the handle 1101 wherein the vertical plane between these two components can be set at an angle relative to each other.

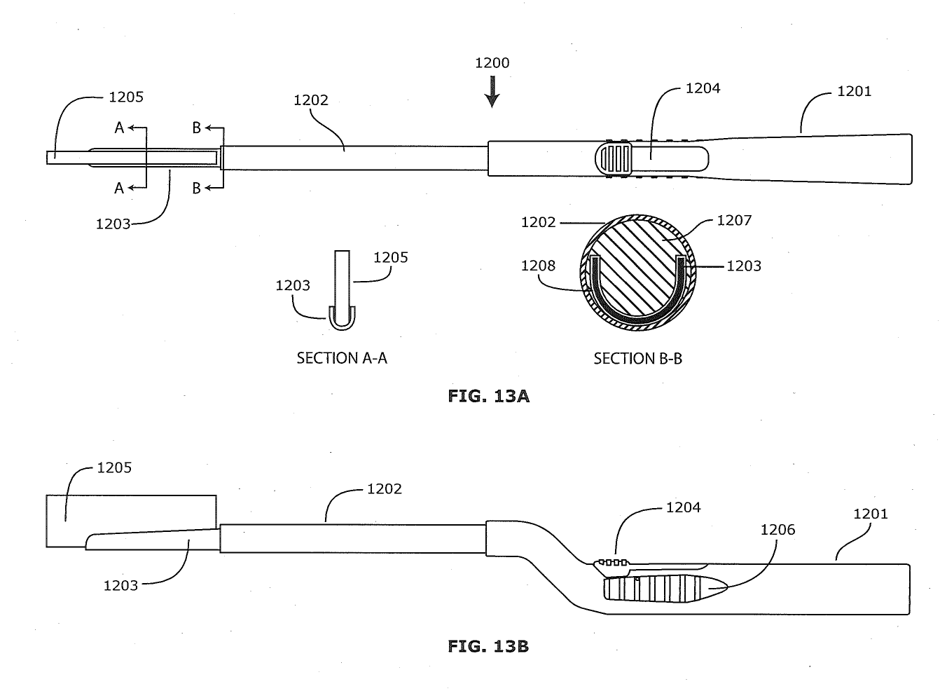

FIG. 13A illustrates an alternative embodiment of the device of the invention 1200 comprising a handle 1201, an elongate member 1202, a shuttle 1203, an actuator 1204, and a packing material 1205. Packing material 1205 may be any of those previously described herein, but may preferably be a xerogel formulated as disclosed in co-pending U.S. patent application 61/259,564, the disclosure of which is incorporate by reference in its entirety. Handle 1201 further comprises a ridged and/or contoured section 1206. The materials of fabrication and means of joining the components of embodiment of the invention 1200 that are common to those of embodiment of the invention 1100 are identical to those previously described for embodiment of the invention 1100. Components of embodiment of the invention 1200 that are unique to embodiment of the invention 1200, such as shuttle 1203, are described in sufficient detail such that one of skill in the art can comprehend their assembly and function. For example, shuttle 1203 may be fabricated from metallic or non-metallic materials known to the art including, but not limited to, aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations, copolymers, and/or alloys thereof, and the like. Section A-A depicts an end-on view of a proximal slice of shuttle 1203 and packing material 1205. As seen in section A-A, shuttle 1203 is generally U-shaped in cross-section and sized to contain packing material 1205. Shuttle 1203 may preferably be sized to apply a compressive load on the outer surfaces of packing material 1205. The outer top edges of shuttle 1203 may be formed such that they are folded-in or curved-in (not shown) wherein the gap or opening between the top opposite edges is less than the widest gap or opening between the walls below the top edge. The internal surfaces of shuttle 1203 (i.e. those that contact the outer surfaces of packing material 1205) may have features (not shown) that secure the packing material 1205 and prevent from inadvertent dislodgement from the shuttle 1203 prior to deployment. For example, these features may include, but are not limited to, texturing, beading, protrusions such as detents, barbs, and the like, ridges, areas of differing material properties such as dimples fabricated from a lower durometer material than the majority of shuttle 1203, combinations thereof, and the like. Section B-B depicts an end-on view of the distal portion of elongate member 1202, further comprising a cap 1207. Cap 1207 may be fabricated from metallic or non-metallic materials known to the art including, but not limited to, aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations, copolymers, and/or alloys thereof, and the like. Cap 1207 may be joined to the distal end of elongate member 1202 using means known to the art including, but not limited to, adhesive bonding, welding, ultrasonic welding, overmolding, threading and tapping, mechanical fixation, friction or interference fits, combinations thereof, and the like. Alternatively, cap 1207 may be an integral part of the elongate member 1202 that is formed or cut using processes known in the art such as stamping, laser cutting and the like and folded in to the lumen opening of the elongate member 1202. Cap 1207 further comprises an opening 1208 that is sized and shaped to slidably accept shuttle 1203 as shown in section B-B.

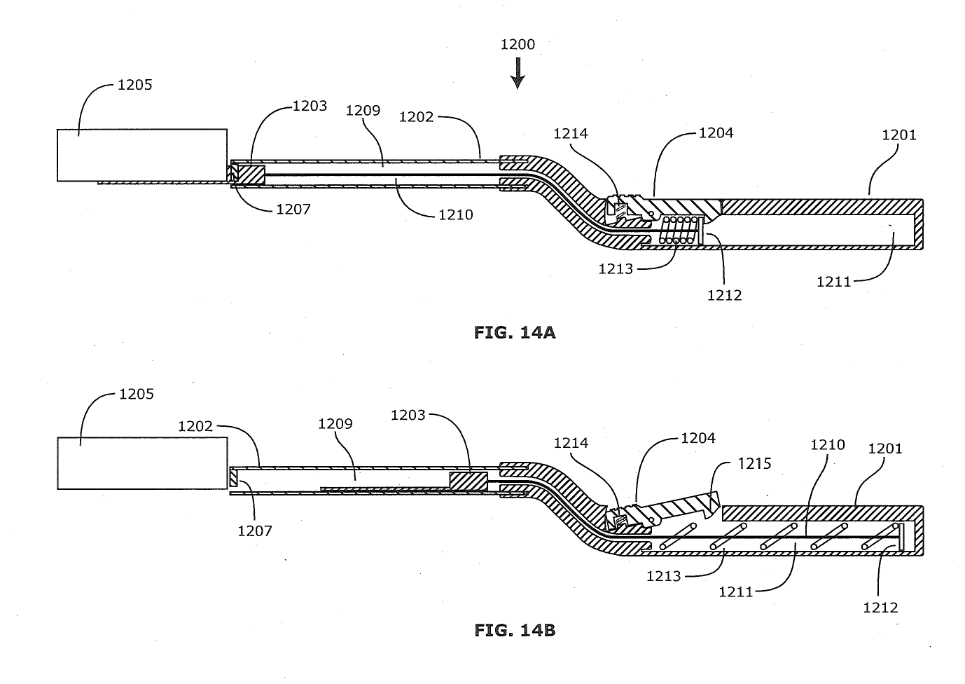

FIGS. 14A and 14B depict embodiment of the invention 1200 with actuator 1204 in the neutral and depressed states, respectively. As can be seen in FIG. 14A, elongate member 1202 further comprises a lumen 1209 and is joined to the distal end of handle 1201 using means known to the art, including but not limited to adhesive bonding, welding, ultrasonic welding, overmolding, threading and tapping, mechanical fixation, friction or interference fits, combinations thereof, and the like. Handle 1201 further comprises an internal space 1211 that is in communication with lumen 1209 of elongate member 1202. Link member 1210 is slidably disposed within lumen 1209 of elongate member 1202 and extends into the internal space 1211 of handle 1201. The distal end of link member 1210 is joined to the proximal end of shuttle 1203 using means known to the art, including but not limited to adhesive bonding, welding, ultrasonic welding, overmolding, threading and tapping, mechanical fixation, friction or interference fits, combinations thereof, and the like. Link member 1210 is flexible to enable translation of link member 1210 through the S-shaped distal portion of handle 1201. The proximal end of link member 1210 is joined to disk 1212 using means known to the art, including but not limited to adhesive bonding, welding, ultrasonic welding, overmolding, threading and tapping, mechanical fixation, friction or interference fits, combinations thereof, and the like. Spring 1213 is disposed within internal space 1211 of handle 1201 and about the proximal portion of link member 1210. Spring 1213 is compressed such that the proximal end of spring 1213 abuts the distal face of disk 1212. Spring 1213 is prevented from expanding to its unloaded length by the interaction between the proximal latch 1215 of the actuator 1204 and the proximal face of disk 1212. Actuator 1204 is biased into a position such that the proximal latch 1215 restricts the proximal motion of disk 1212 by spring 1214.

As shown in FIG. 14B, the action of depressing the distal portion of actuator 1204 with sufficient force to overcome the resisting force of spring 1214 raises the proximal latch 1215 of actuator 1204. When the proximal latch 1215 is moved out of contact with the proximal face of disk 1212, spring 1213 expands to its unloaded position (i.e. the proximal end of spring 1213 translates proximally within internal space 1211) and drives disk 1212 to the proximal portion of internal space 1211. The proximal motion of disk 1212 is transmitted through link member 1210 to shuttle 1203. The proximal motion of shuttle 1203 relative to elongate member 1202 pulls packing material 1205 in the proximal direction. The proximal end of packing material 1205 contacts the distal face of cap 1207 and the distal end of elongate member 1202, halting the proximal motion of packing material 1205. Shuttle 1203 continues to translate in the proximal direction, releasing packing material 1205 from device 1200. Alternatively (not shown), apparatus 1200 can be configured such that the spring 1213 is located at the proximal side of disk 1212 wherein its proximal end is affixed or anchored to the proximal end of handle 1101. Load to the spring 1213 is applied by stretching the distal end until disk 1212 engages to latch 1215 of actuator 1204. Other types of spring 1213 known in the art may also be used such as constant force spring, elastic rubber and the like. The previously described considerations for embodying a re-usable version of the device of the invention 1100 apply to device of the invention 1200 as well.

Apparatus 1200 may also have added feature such as an adjustable elongate member 1202 wherein the elongate member 1202 can be rotated along its longitudinal central axis (not shown). This feature allows adjustment of the packing material 1205 to vary its position relative to the handle 1201 wherein the vertical plane between these two components can be set at an angle relative to each other.

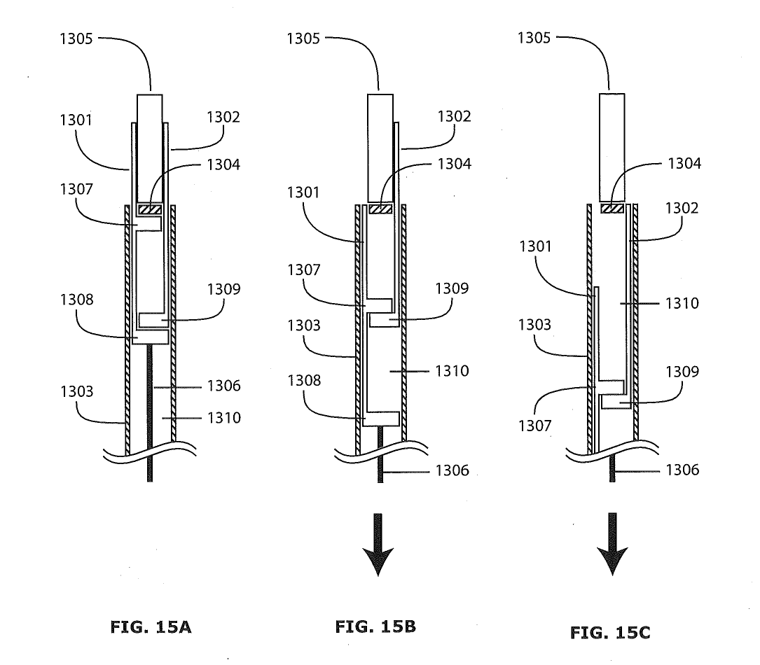

FIGS. 15A-15C depict an alternative means for releasing or delivering a packing material into the nasal cavity. In these figures, force applied in the proximal direction is denoted by a heavy black arrow; the means of applying such force may be those previously disclosed in this specification or any other means within the art. FIG. 15A depicts a shuttle assemble comprised of a primary tine 1301 and a secondary tine 1302, an elongate member 1303, a cap 1304, a packing material 1305, and a link member 1306. Packing material 1305 may be any of those previously described herein, but may preferably be a xerogel formulated as disclosed in co-pending U.S. patent application 61/259,564, the disclosure of which is incorporate by reference in its entirety. Primary tine 1301 further comprises a primary flange 1307 and secondary flange 1308, while secondary tine 1302 further comprises tertiary flange 1309. Elongate member 1303 further comprises a lumen 1310. Primary and secondary tines 1301 and 1302, and primary, secondary, and tertiary flanges 1307, 1308, and 1309 may be fabricated from metallic or non-metallic materials known to the art, including but not limited to aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin combinations, copolymers, and/or alloys thereof, and the like. As previously discussed for embodiment of the invention 1200, the internal surfaces of the distal portions of primary tine 1301 and secondary tine 1302 may further comprise features that secures the packing material 1305 and prevent from inadvertent dislodgement from primary and secondary tines 1301 and 1302, respectively, prior to deployment. Primary flange 1307 and secondary flange 1308 may be joined to primary tine 1301 using means known to the art including, but not limited to, adhesive bonding, welding, ultrasonic welding, overmolding, threading and tapping, mechanical fixation, friction or interference fits, cold working, combinations thereof, and the like. Tertiary flange 1309 may be joined to secondary tine 1302 using means known to the art including, but not limited to, adhesive bonding, welding, ultrasonic welding, overmolding, threading and tapping, mechanical fixation, friction or interference fits, cold working, combinations thereof, and the like. Alternatively, flanges 1307, 1308, and 1309 may be an inherent feature of their respective tines 1301 or 1302. For example, a tine may be formed by bending a length of stainless steel such that a flange is formed in the body of the tine. Elongate member 1303 may be fabricated from metallic or non-metallic materials known in the art including, but not limited to aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like. Cap 1304 may be fabricated from materials known in the art including, but not limited to aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like and joined to the distal end of elongate member 1303 using means known to the art including, but not limited to adhesive bonding, welding, ultrasonic welding, overmolding, threading and tapping, mechanical fixation, friction or interference fits, cold working, combinations thereof, and the like. Alternatively, cap 1304 may be an integral part of the elongate member 1303 that is formed or cut using processes known in the art such as stamping, laser cutting and the like and folded in to the lumen opening of the elongate member 1303. Link member 1306 may be fabricated from materials known to the art including, but not limited to aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like. The distal end of link member 1302 may be joined to the proximal portion of secondary flange 1308 using means known to the art including, but not limited to adhesive bonding, welding, ultrasonic welding, overmolding, threading and tapping, mechanical fixation, friction or interference fits, cold working, combinations thereof, and the like. Primary tine 1301 and secondary tine 1302 are slidably disposed within lumen 1310 of elongate member 1303 and arranged such that the distal ends of primary tine 1301 and secondary tine 1302 extend distally beyond the distal end of elongate member 1303.

FIGS. 15B and 15C illustrate the action of the components as force is applied to link member 1306 in the proximal direction. The proximal translation of link member 1306 within the lumen 1310 of elongate member 1303 is transmitted to primary tine 1301, which in turn translates in the proximal direction until the proximal face of primary flange 1307 contacts the distal face of tertiary flange 1309. At this point, the distal portion of primary tine 1301 has retracted away from packing material 1305 and into the lumen 1310 of elongate member 1303. Further application of force in the proximal direction to link member 1306 continues to pull primary tine 1302 and secondary tine 1302 in the proximal direction, retracting distal portion of secondary tine 1302 away from packing material 1305 and into the lumen 1310 of elongate member 1303. Any proximal motion of packing material 1305 that may occur during the proximal translation of link member 1306 is halted due to interference with the distal face of cap 1304 and/or the distal end of elongate member 1303. Though this example is provided with two tines, it should be clear to one of skill in the art that any number or arrangement of tines, or sequence in retracting any number or arrangement of tines, is contemplated.

That which is claimed:

1. A device for use in positioning a material in a nasal cavity of a subject, comprising;
a handle having a distal end, a proximal end and a channel wherein the handle is disposed at the proximal end of the device;
an actuator slidably disposed on the handle;
an elongate member having a distal end, a proximal end and at least one lumen, wherein the proximal end is joined to the distal end of the handle;
a shuttle having a distal end and a proximal end slidably disposed on the elongate member;
a link member having a distal end and a proximal end slidably disposed on the elongate member and the handle, wherein the distal end of the link member is joined to the shuttle and the proximal end is joined to the actuator.

2. The device of claim 1, wherein movement of the actuator along the handle is directly translated to the shuttle via the link member.

3. The device of claim 1, wherein the shuttle comprises at least two tines.

4. The device of claim 1, wherein the shuttle comprises a shape that is U-shaped in cross section.

5. The device of claim 1, wherein the shuttle extends beyond the distal end of the elongate member

6. The device of claim 1, wherein a portion of the actuator is disposed within the handle and a portion of the actuator is external to a surface of the handle.

7. The device of claim 1, wherein a longitudinal axis of the elongate member is offset from a longitudinal axis of the handle.

8. A device for use in positioning a material in a nasal cavity of a subject, comprising;
a handle having a distal end, a proximal end and a channel wherein the handle is disposed at the proximal end of the device;
an actuator disposed on the handle;
a disk slidably disposed in the channel of the handle;
an elongate member having a distal end, a proximal end and at least one lumen, wherein the proximal end of the elongate member is joined to the distal end of the handle;
a shuttle having a distal end and a proximal end slidably disposed on the elongate member;
a link member having a distal end and a proximal end slidably disposed on the lumen of the elongate member and the channel of the handle, wherein the distal end of the link member is joined to the proximal end of the shuttle and the proximal end is joined to the disc; and
a spring disposed in the channel of the handle and on the link member, wherein the proximal end of the spring is positioned against the disk.

9. The device of claim 8, wherein a longitudinal axis of the handle is offset from a longitudinal axis of the elongate member.

10. The device of claim 8, wherein the distal end of the handle has a shape comprising of at least one bend.

11. The device of claim 8, wherein the actuator further comprises a latch extending in the channel of the handle for engaging with the disk.

12. The device of claim 11, wherein the actuator further comprises a spring that maintains the latch in a biased position that keeps the actuator latch engaged with the disk.

13. The device of claim 12, wherein pressing the actuator disengages the disk from the actuator latch.

14. The device of claim 8, wherein the disk further comprises an edge that keeps the latch of the actuator in an engaged position.

15. The device of claim 8, wherein the link member comprises a flexible material.

16. The device of claim 8, wherein the shuttle further comprises at least two tines.

17. The device of claim 16, wherein the shuttle comprises two tines for positioning a material there between.

18. The device of claim 8, wherein the shuttle comprises a shape that is U-shaped in cross section.

19. The device of claim 18, wherein the shuttle further comprises an opening sized for holding a material.

20. The device of claim 8, wherein the shuttle further comprises a post disposed at the proximal end of the shuttle.

21. The device of claim 8, wherein the elongate member comprises a cap disposed at the distal end of the elongate member.

22. The device of claim 21, wherein the cap further comprises an opening to slidably receive the shuttle and maintain orientation of the shuttle relative to the elongate member and the handle.

23. The device of claim 8, wherein the spring exerts a force against the disk when the disk is engaged on the latch of the actuator.

24. The device of claim 23, wherein the force exerted by the spring shifts the disk from a first position to a second position when the disk is disengaged from the latch of the actuator.

25. The device of claim 24, wherein the movement of the disk from the first position to the second position is directly translated to the shuttle.

26. The device of claim 24, wherein movement of the shuttle from the first position to the second position releases the material.

27. A device for use in positioning a material in a nasal cavity or a subject, comprising;
an elongate member having a distal end, a proximal end, a lumen, and at least one sidewall port;
a flange disposed at the proximal end of the elongate member;
an extension line disposed at a proximal end of the device, wherein the extension line comprises a distal end, a proximal end, and a lumen, wherein the proximal end of the extension line is joined to the distal end of the elongate member,
a connection port disposed at the proximal end of the extension line,
an expandable member disposed at the distal end of the elongate member and joined to the elongate member between the distal end and proximal end of the elongate member such that a space bounded by the internal surface of the expandable member and external surface of the elongate member is in fluid communication with the injection ports of the elongate member; and
a material disposed within the expandable member.

28. The device of claim 27, wherein the lumen of the extension line is in fluid communication with the lumen of the elongate member.

29. The device of claim 27, wherein the packing material is secured in place.

30. The device of claim 29, wherein a weld secures the packing material in place.

31. The device of claim 29, wherein a weave, braid, or suture secures the packing material in place.

32. The device of claim 27, wherein infusion of a fluid into the connection port expands the expandable member.

33. The device of claim 32, wherein expansion of the expandable member releases the packing material from the apparatus.

34. A delivery system comprising a nasal packing material and the device of claim 1.

35. The delivery system of claim 34, wherein the nasal packing material is positioned within the shuttle prior to use.

36. The delivery system of claim 27, wherein the nasal packing material is provided separately from the device of claim 1 and positioned in the shuttle prior to use by an operator.

37. A delivery system comprising a nasal packing material and the device of claim 8.

38. The delivery system of claim 37, wherein the nasal packing material is positioned within the shuttle prior to use.

39. The system of claim 37, wherein the nasal packing material is provided separately from the device of claim 11 and positioned in the shuttle prior to use by the operator.

40. A delivery system comprising a nasal packing material and the device of claim 27.

41. The delivery system of claim 40, wherein the nasal packing material is positioned within the expandable member prior to use.

42. The system of claim 40, wherein the nasal packing material is provided separately from the device of claim 27 and positioned in the expandable member prior to use by the operator.

43. A method of treating epistaxis comprising;
advancing a distal end of the elongate member of the device of claim 26 into a nostril of a patient until the flange contacts the alar rim and/or the columella;
expanding the expandable member from a first diameter to a second diameter;
releasing the nasal packing from the apparatus;
applying the nasal packing to the site of bleeding;
maintaining the expandable member at the second diameter to compress and apply pressure to the nasal packing against the site of bleeding;
collapsing the expandable member from the second diameter to the first diameter; and
removing the apparatus from the nostril of the patient.

44. A method of delivering a nasal packing material to a target nasal anatomical space of a subject, comprising;
advancing a distal end of the device of claim 1 into a nostril of a subject, placing the shuttle containing a nasal packing material into a target nasal anatomical space;
retracting the actuator in the proximal direction to release the nasal packing material in the target nasal anatomical space, and
removing the apparatus from the nostril of the subject.

45. The method of claim 44, wherein the target nasal anatomical space is the middle meatus.

46. The method of claim 44, wherein the target nasal anatomical space is the frontal sinus ostium.

47. The method of claim 44, wherein the target nasal anatomical space is the maxillary sinus ostium.

48. The method of claim 44, wherein the target nasal anatomical space is an ethmoid cell.

49. The method of claim 44, wherein the target nasal anatomical space is the sphenoid sinus ostium.

50. A method of delivering a nasal packing material to a target nasal anatomical space of a subject, comprising;
advancing a distal end of the device of claim 8 into a target nasal anatomical space of a subject;
placing the shuttle containing the nasal packing material into the target nasal anatomical space;
activating the actuator to release the nasal packing in the target nasal anatomical space; and
removing the apparatus from the nostril of the subject.

51. The method of claim 50, wherein the target nasal anatomical space is the middle meatus.

52. The method of claim 50, wherein the target nasal anatomical space is the frontal sinus or frontal sinus ostium.

53. The method of claim 50, wherein the target nasal anatomical space is the maxillary sinus or maxillary sinus ostium.

54. The method of claim 50, wherein the target a nasal anatomical space is an ethmoid cell.

55. The method of claim 50, wherein the target nasal anatomical space is the sphenoid sinus or sphenoid sinus ostium.
